(19) United States
(12) Patent Application Publication
(10) Pub. No.: US 2023/0284601 A1
(43) Pub. Date: Sep. 14, 2023

(54) METHOD FOR CONTROLLING HATCHING OF BLACK SOLDIER FLY EGGS

(71) Applicant: Protix B.V., Dongen (NL)

(72) Inventors: Ward Tollenaar, Dongen (NL); Lotte Joosten, Dongen (NL); Eric Holland Schmitt, Dongen (NL)

(73) Assignee: Protix B.V., Dongen (NL)

(21) Appl. No.: 17/792,152

(22) PCT Filed: Dec. 17, 2020
(86) PCT No.: PCT/NL2020/050799

§ 371 Date: Jul. 12, 2022

(30) Foreign Application Priority Data
Jan. 17, 2020 (NL) 2024692

(51) Int. Cl. A01K 67/033

(52) U.S. Cl. CPC A01K 67/033; A01K 2227/706

(57) ABSTRACT

The invention relates to a method for controlling hatching of Black soldier fly eggs, comprising the steps of incubating the eggs at a selected temperature for a selected time period; cooling the incubated eggs at a temperature lower than the selected temperature, starting at a selected time point; warming the eggs again at the selected temperature for a further selected period, up till the eggs hatch. Furthermore, the invention relates to a batch of Black soldier fly eggs kept at the temperature at which the eggs are cooled according to the invention, which cooled eggs are capable of hatching once warmed at the selected temperature according to the invention for a predetermined period of time. This way the invention provides for a method for delaying of hatching of an egg. Furthermore, the method of the invention and the batch of cooled eggs of the invention provide a method for synchronizing of hatching of a first egg and a second egg which are deposited at a first time point and at a second time point. In addition, the method of the invention and the batch of cooled eggs of the invention allow for compressing the time window for hatching of a batch of eggs which are deposited within a time frame of e.g. between 0 hours and 12 hours, to a time period of 14 hours or less.

### TECHNICAL FIELD

The invention relates to a method for controlling hatching of Black soldier fly eggs, comprising the steps of incubating the eggs at a selected temperature for a selected time period; cooling the incubated eggs at a temperature lower than the selected temperature, starting at a selected time point; warming the eggs again at the selected temperature for a further selected period, up till the eggs hatch. Furthermore, the invention relates to a batch of Black soldier fly eggs kept at the temperature at which the eggs are cooled according to the invention, which cooled eggs are capable of hatching once warmed at the selected temperature according to the invention for a predetermined period of time. This way the invention provides for a method for delaying of hatching of an egg. Furthermore, the method of the invention and the batch of cooled eggs of the invention provide a method for synchronizing of hatching of a first egg and a second egg which are deposited at a first time point and at a second time point. In addition, the method of the invention and the batch of cooled eggs of the invention allow for compressing the time window for hatching of a batch of eggs which are deposited within a time frame of e.g. between 0 hours and 12 hours, to a time period of 14 hours or less.

### BACKGROUND

Insects are considered one of the most promising means for protein and for organic residual recovery. Prominent examples of species proposed for the indicated applications include the Black soldier fly ( Hermetia illucens L.), the house fly ( Musca domestica L.), and the mealworm ( Tenebrio molitor L.).

Methods improving the efficiency of insect farming relating to improvements in timely farming colonies of insects having essentially the same and predetermined age are particularly valuable for large scale production. This, because of the batch wise nature of the insect farming steps that should be performed in order to be able to arrive at an economically viable scale. Since aiming for large-scale insect farming is a desired industrial activity that involves live animals, synchronization of the age of insects in a colony and controlling the age of colonies at a predetermined point in time, which insects are then essentially in the same stage of the insect life cycle at a desired moment relating to for example processing capacity in a factory, would contribute to efficient use of farming facilities and insect processing facilities, and would aid in achieving predictable production volumes at predicted time points during the day or week, etc. Furthermore, timing, synchronization and steering of the age of batches of insect colonies which are in subsequent insect stages would further contribute to efficient use of farming facilities and insect processing facilities.

However, methods and means for efficaciously and beneficially interfering in and controlling of the life cycle of insects forming a colony, in particular Black soldier flies, such that within the colony the insects essentially have the same age at a desired moment in time, wherein the colony has a predetermined and desired size, to the benefit of industrial-scale insect farming and insect product manufacturing, are at present not available in the art, and in particular not for Black soldier fly farming.

Therefore, a solution still needs to be found that allows for precisely controlling the age of a batch of insects at a predetermined and desired time point and for controlling and synchronizing the age variation within a selected batch of insects.

### SUMMARY

For embodiments of the present invention, it is a first goal to provide an improved method for industrial scale insect farming.

It is one of several objectives of embodiments of the current invention to provide a solution to the problem of providing colonies of insects at a predetermined moment in time, wherein the insects have a predetermined age, and/or wherein the insect colony has a predetermined size.

At least one of the above objectives of embodiments of the invention is achieved by providing a method for controlling the time-point of hatching of insect eggs. At least one of the above objectives of embodiments of the invention is also achieved by providing a method for controlling the time-window in which insect eggs of a selected age hatch. At least one of the above objectives of embodiments of the invention is further achieved by providing a method for synchronizing the age of insects in a colony.

The present invention will be described with respect to particular embodiments but the invention is not limited thereto but only by the claims. The embodiments of the invention described herein can operate in combination and cooperation, unless specified otherwise.

In brief, the invention relates to a method for controlling hatching of Black soldier fly eggs, comprising the steps of incubating the eggs at a selected temperature for a selected time period; cooling the incubated eggs at a temperature lower than the selected temperature, starting at a selected time point; warming the eggs again at the selected temperature for a further selected period, up till the eggs hatch. Furthermore, the invention relates to a batch of Black soldier fly eggs kept at the temperature at which the eggs are cooled according to the invention, which cooled eggs are capable of hatching once warmed at the selected temperature according to the invention for a predetermined period of time. This way, the invention provides for a method for delaying of hatching of an egg. Furthermore, the method of the invention and the batch of cooled eggs of the invention provide a method for synchronizing of hatching of a first egg and a second egg which are deposited at a first time point and at a second time point. In addition, the method of the invention and the batch of cooled eggs of the invention allow for compressing the time window for hatching of a batch of eggs which are deposited within a time frame of e.g. between 0 hours and 12 hours, to a time period of 14 hours or less.

A first aspect of the invention relates to a method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6. Reference is made to Scheme I and Scheme II.

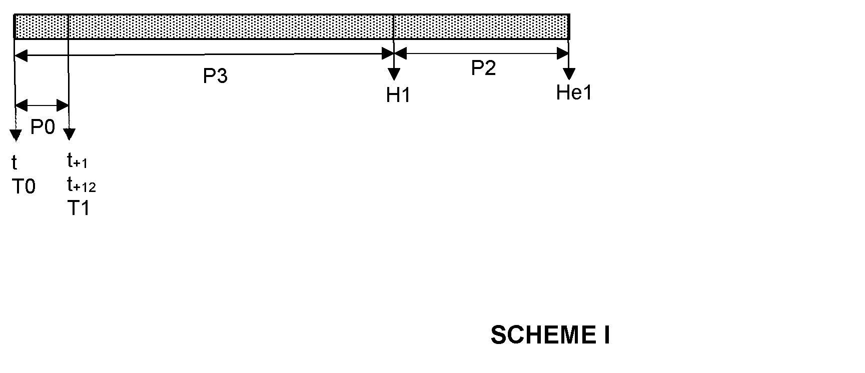

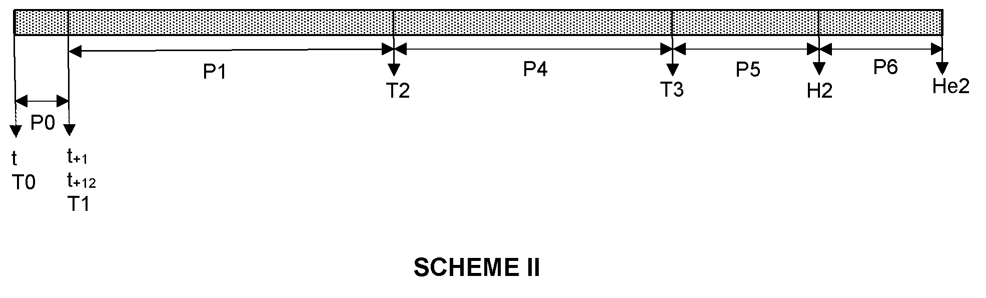

Preferred is the method of the invention, wherein relative humidity during any one or more of P0, P1, P2, P3, P4, P5 and P6, preferably during all of P0, P1, P2, P3, P4, P5 and P6 is controlled and is kept at a predetermined value, preferably between 65% and 100% RH.

An aspect of the invention relates to a batch of Black soldier fly eggs comprising embryos and kept at a temperature of between 0° C. and below 14° C. with a relative humidity of 65% or higher, wherein the embryos are capable of developing into Black soldier fly neonates if the temperature is risen to 14° C. - 40° C. while the relative humidity remains 65% or higher.

Preferred is the batch of Black soldier fly eggs comprising embryos according to the invention, wherein the embryos are capable of developing into Black soldier fly neonates if subjected to subsequent steps d) and e) of the method according to the invention.

Preferred is the method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, wherein the BSF embryos are older than 2-5 hr of age when the cooling step c) starts at T2, and the embryos are younger than 33 hr of age or older than 36 hr of age, at the start of the cooling step c) at T2, when the BSF eggs are incubated at 30° C. and 80% relative humidity (RH), since T1 and up to T2, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6.

Preferred is the method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6, wherein the selected controlled second temperature is between 4° C. and below 14° C., preferably 5° C. - 10° C.

Preferred is the method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6, wherein the selected first incubation period P1 and the third incubation period P5 together are the same or longer than time period P3, preferably between 1 hour and 10 hours longer, more preferably 2 hours - 6 hours, most preferably 3 hours - 4 hours.

Preferred is the method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly (BSF) eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, wherein the BSF embryos are older than 2-5 hr of age when the cooling step c) starts at T2, and the embryos are younger than 33 hr of age or older than 36 hr of age, at the start of the cooling step c) at T2, when the BSF eggs are incubated at 30° C. and 80% relative humidity (RH), since T1 and up to T2, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6, wherein the selected first incubation period P1 and the third incubation period P5 together are the same or longer than time period P3, preferably between 1 hour and 10 hours longer, more preferably 2 hours - 6 hours, most preferably 3 hours - 4 hours.

Preferred is the method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6, wherein the selected controlled second temperature is between 4° C. and below 14° C., preferably 5° C. - 10° C., and wherein the selected first incubation period P1 and the third incubation period P5 together are the same or longer than time period P3, preferably between 1 hour and 10 hours longer, more preferably 2 hours - 6 hours, most preferably 3 hours - 4 hours.

Preferred is the method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly (BSF) eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, wherein the BSF embryos are older than 2-5 hr of age when the cooling step c) starts at T2, and the embryos are younger than 33 hr of age or older than 36 hr of age, at the start of the cooling step c) at T2, when the BSF eggs are incubated at 30° C. and 80% relative humidity (RH), since T1 and up to T2, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6, wherein the selected first incubation period P1 and the third incubation period P5 together are the same or longer than time period P3, preferably between 1 hour and 10 hours longer, more preferably 2 hours - 6 hours, most preferably 3 hours - 4 hours, wherein the selected controlled second temperature is between 4° C. and below 14° C., preferably 5° C. - 10° C.

Preferred is the method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6, wherein time point T2 for starting cooling of the batch of eggs is at least 2 hours after time point T0 at which the first eggs of the batch of eggs are deposited, and at least 3 hours before yolk in the eggs is absent in the cranial side of embryos in the eggs or at least 3 hours after yolk in the eggs is absent in the cranial side of the embryos in the eggs.

Preferred is the method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6, wherein time point T2 for starting cooling of the batch of eggs is at least 2 hours after time point T0 at which the batch of eggs is deposited, and at least 2 hours before dorsal closure in embryos in the eggs or at least 1 hour after dorsal closure in the embryos in the eggs, preferably 2 hours - 32 hours before dorsal closure in the embryos in the eggs, more preferably 3 hours - 20 hours before dorsal closure in the embryos in the eggs or 1 hour - 25 hours after dorsal closure in the embryos in the eggs, most preferably 3 hours - 22 hours after dorsal closure in the embryos in the eggs.

Preferred is the method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6, wherein in step c) T2 is selected from a time point between 25 hours and 0 hour before time point H1 of step bb), preferably between 20 hours and 0 hour, more preferably between 18 hours and 0 hour, most preferably between 16 hours and 0 hour, such as selected from 14 hours - 2 hours or 12 hours - 4 hours.

### DEFINITIONS

The term “ovisite” has its regular scientific meaning throughout the text, and here refers to a device designed to collect eggs. Typically, an ovisite has dimensions of between about 15 cm and 60 cm (width) times between about 10 cm and 40 cm (height) times between about 0.6 cm and 2.4 cm (depth), such as an ovisite of about 30 cm (width) times about 20 cm (height) times about 1.2 cm (depth). A preferred ovisite for use in the method of the invention, is an ovisite with for example honeycomb architecture comprising for example hexagonal openings, such as a cardboard honeycomb. Such cardboard honeycomb comprises sufficient and enough space for bearing a number of insect eggs, which number is sufficiently high for provision of a batch of BSF eggs for use in the method of the invention.

The term “batch” is here defined as a number of insect eggs that has been deposited typically in an ovisite or in a number of ovisites together. A batch of insect eggs, contained in an ovisite, may comprise insect eggs which have essentially the same age, and may comprise insect eggs which may have a maximum age difference essentially equal to the time an ovisite has been exposed to gravid

BSF female flies, e.g. inside a cage. A batch of BSF eggs typically consists of between 50 gr of eggs (for example when a single ovisite is considered) and 2.000 kg of eggs (for example when a multitude of ovisites is considered).

The term “neonate”, or “neonate larvae”, has its regular scientific meaning throughout the text, and here refers to a newborn insect larva, such as newborn BSF larvae, for example less than 1 hour of age post hatching.

The term “cool”, or “cooling”, has its regular scientific meaning throughout the text, and here refers to (cooling at) a temperature of 17° C. or lower, and typically a temperature of between 0° C. and 16° C. or below, such as between 0° C. and lower than 14° C.

The term “depositing”, or “deposit”, or “deposited”, in the context of an insect egg, has its regular scientific meaning throughout the text, and here refers to the laying of an egg by a gravid female insect such as a gravid female BSF. Typically, the eggs are deposited in an ovisite.

The term “hatching” has its regular scientific meaning throughout the text, and here refers to the incubation of an insect egg such as a fertilized BSF egg, until a neonate insect larva emerges from the egg. In the context of the invention, insect larvae hatch from the incubated eggs upon incubation of deposited eggs at a suitable temperature and relative humidity for a suitable time.

### BRIEF DESCRIPTION OF THE DRAWINGS

FIG. 1. Influence of cooling of Black Soldier Fly (BSF) embryos for 24 hr at an age of between 0 hours and 58 hours, on the extent of hatching of the BSF eggs after the cool phase.

FIG. 1A. Overview of hatching success for cooling of eggs at different embryo ages ranging from 0-1 hr to 57-58 hr; the boxplots represent the average hatching success of combined six ovisites for experimental treatments (embryos subjected to a cooling step of 24 h at 10° C.) and the average hatching success of combined twelve ovisites for the control (embryos not subjected to a cooling step; continuous incubation of eggs at 30° C. and 80% RH; hatching measured as weight decrease of the ovisites during 24 h after a 60 hr warm period at 30° C., and set at 100% for the control). Below the graph, for each time point of embryo age at which the cooling step started, from 0 hr till 55 hr of age, a representative photo of a BSF embryo is depicted.

FIG. 1B. Overview according to FIG. 1A, of hatching success for cooling of eggs at different embryo ages ranging from 0-1 hr to 57-58 hr. The time window of 12 hours for embryos in eggs in ovisites collected and incubated at 30° C. for 42 hours (embryos 42-43 hours of age) before the start of the cooling up to ovisites collected and incubated at 30° C. for 54 hours (embryos 54-55 hours of age) before the start of the cooling, is indicated with ‘← 12 hr →’: this is a suitable time frame for cooling eggs which have an age difference of up to 12 hours, when subsequent hatching success compared to control is considered, once ovisites are warmed again to 30° C. The sign ‘← C →’ indicates the time window for embryos at an age of 35 till 58 hours of age, at which the cooling step can start in order to obtain about 75% hatching eggs or higher after 58 hours of incubation at 30° C. in total. The ‘dr’ signs indicate the 25% weight loss of the ovisites independent of hatching eggs between 0 hr and 34 hr after hatching and the start of the cooling phase. Embryos 0-1 hr of age at the start of the cooling phase did not hatch (about 25% weight loss relates most likely to drying of the non-vital eggs/embryos). Similarly, embryos 33-34 hr of age at the start of the cooling phase did not hatch (about 25% weight loss relates most likely to drying of the non-vital eggs/embryos). Embryos 3-4 hr of age up to 30-31 hr of age at the start of the cooling phase hatched to some extent when warmed to 30° C. again. When the cooling phase started with embryos 36-37 hr of age or older, relative high numbers of neonates were obtained, compared to embryos for which the cooling phase started at 3-4 hr - 30-31 hr of age, and hatching rate was consistently high, without signs of weight loss of ovisites due to drying of embryos/eggs (near-100% hatching compared to control).

FIG. 1C. Representative BSF embryo 0-1 hours of age. Eggs do not hatch when embryos 0-1 hr of age are cooled and again warmed.

FIG. 1D. Representative BSF embryo 3-4 hours of age.

FIG. 1E. Representative BSF embryo 6-7 hours of age.

FIG. 1F. Representative BSF embryo 9-10 hours of age.

FIG. 1G. Representative BSF embryo 12-13 hours of age. ‘d’ refers to the dorsal side of the embryo. ‘v’ refers to the ventral side of the embryo.

FIG. 1H. Representative BSF embryo 15-16 hours of age.

FIG. 1I. Representative BSF embryo 18-19 hours of age.

FIG. 1J. Representative BSF embryo 21-22 hours of age.

FIG. 1K. Representative BSF embryo 24-25 hours of age. ‘c’ refers to the cranial part of the embryo; ‘y’ refers to yolk; ‘E’ refers to an eye of the embryo.

FIG. 1L. Representative BSF embryo 27-28 hours of age.

FIG. 1M. Representative BSF embryo 30-31 hours of age.

FIG. 1N. Representative BSF embryo 33-34 hours of age. Eggs do not hatch when embryos 33-34 hr of age are cooled and again warmed. ‘h’ refers to the head of the embryo.

FIG. 1O. Representative BSF embryo 36-37 hours of age. Eggs efficiently hatch when embryos 36-37 hr of age are cooled and subsequently warmed again. The yolk is not present anymore at the cranial side; only present at the dorsal side of the embryo.

FIG. 1P. Representative BSF embryo 39-40 hours of age. Eggs efficiently hatch when embryos 39-40 hr of age are cooled and subsequently warmed again. ‘-M’ refers to the absence of the mouth part of the head of the embryo.

FIG. 1Q. Representative BSF embryo 42-43 hours of age. Eggs efficiently hatch when embryos 42-43 hr of age are cooled and subsequently warmed again.

FIG. 1R. Representative BSF embryo 45-46 hours of age. Eggs efficiently hatch when embryos 45-46 hr of age are cooled and subsequently warmed again. ‘M’ refers to the visible mouth part of the head of the embryo.

FIG. 1S. Representative BSF embryo 48-49 hours of age. Eggs efficiently hatch when embryos 48-49 hr of age are cooled and subsequently warmed again.

FIG. 1T. Representative BSF embryo 51-52 hours of age. Eggs efficiently hatch when embryos 51-52 hr of age are cooled and subsequently warmed again. ‘T’ refers to trachea of the embryo.

FIG. 1U. Representative BSF embryo 54-55 hours of age. Eggs efficiently hatch when embryos 54-55 hr of age are cooled and subsequently warmed again.

FIG. 1V. Representative BSF embryo 57-58 hours of age. Eggs efficiently hatch when embryos 57-58 hr of age are cooled and subsequently warmed again.

FIG. 2. The time of hatching to about completion for BSF eggs, which were incubated for 45-58 hours at 30° C. and 80% relative humidity after deposition in ovisites, was determined for embryos which were not (A.), or which were (B.) subjected to a cooling step lasting for 2 hours and starting at t = 58 hr (relating to the oldest embryos; cooling started at 45 hr relating to the youngest embryos), followed by ramping up the temperature back to 30° C. after the 2-hours cooling period. The time of hatching to near completion of BSF eggs, which were incubated for 45-58 hours at 30° C. and 80% relative humidity after being deposited in ovisites, was determined for embryos which were not (C.), or which were (D.) subjected to a cooling step lasting for 6 hours and starting at t = 58 hr (relating to the oldest embryos; cooling started at 45 hr relating to the youngest embryos), followed by ramping up the temperature back to 30° C. after the 6-hours cooling period. The embryos at the time point at which the cooling step started were between 45 hours of age and 58 hours of age. Extent of hatching was followed for 24 hours starting at t = 58 hr (relating to the oldest embryos), at the end of the incubation period of 45-58 hr at 30° C. and 80% relative humidity (A., C.), or extent of hatching was followed for 22 hours starting at t = 60 hr (relating to the oldest embryos), at the end of the incubation period of 45-58 hr at 30° C. and 80% relative humidity, followed by the 2-hours cooling step at 10° C. and the ramp-up step back to 30° C. (B.), or extent of hatching was followed for 18 hours starting at t = 64 hr (relating to the oldest embryos), at the end of the incubation period of 45-58 hr at 30° C. and 80% relative humidity, followed by the 6-hours cooling step at 10° C. and the ramp-up step back to 30° C. (D.). During the measurement of the extent of hatching eggs, temperature was 30° C. at 80% relative humidity.

FIGS. 3A-R. Images of microscope slides of black soldier fly eggs, eggs are displayed with the anterior side to the left and posterior side to the right. The ventral side is on the bottom and the dorsal side on top. Time windows for the stages are in the bottom right corner of each frame. Letters mark morphological landmarks (c = chorion, cm = cellular membrane, e = eye spot, gb = germ band, h = head, if = intersegmental furrow, mp = mouth parts, pf = parasegmental furrow, si = stomodeal invagination, t = trachea, y = yolk). See Example 4 for a detailed description.

FIG. 4. Relative hatch success of chilled eggs at different ages with MARS model fitted over the data (n = 98).

### DETAILED DESCRIPTION

The present invention will be described with respect to particular embodiments but the invention is not limited thereto but only by the claims.

Furthermore, the terms first, second, third and the like in the description and in the claims, are used for distinguishing between similar elements and not necessarily for describing a sequential or chronological order. The terms are interchangeable under appropriate circumstances. The embodiments of the invention can operate in other sequences than described or illustrated herein.

The embodiments of the invention described herein can operate in combination and cooperation, unless specified otherwise.

Furthermore, the various embodiments, although referred to as “preferred” or “e.g.” or “for example” or “in particular” are to be construed as exemplary manners in which the invention may be implemented rather than as limiting the scope of the invention.

The term “comprising”, used in the claims, should not be interpreted as being restricted to the elements or steps listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a method comprising step A and step B” should not be limited to a method consisting only of steps A and B, rather with respect to the present invention, the only enumerated steps of the method are A and B, and further the claim should be interpreted as including equivalents of those steps. Similarly, the scope of the expression “a product (or e.g. a composition) comprising component C and component D” should not be limited to a product consisting only of component C and D, rather with respect to the present invention, the only enumerated components of the product (or composition) are C and D, and further the claim should be interpreted as including equivalents of those components.

In addition, reference to an element by the indefinite article “a” or “an” does not exclude the possibility that more than one of the element are present, unless the context clearly requires that there is one and only one of the elements. The indefinite article “a” or “an” thus usually means “at least one”.

While the invention has been described in terms of several embodiments, it is contemplated that alternatives, modifications, permutations and equivalents thereof will become apparent to one having ordinary skill in the art upon reading the specification and upon study of the drawings. The invention is not limited in any way to the illustrated embodiments. Changes can be made without departing from the scope which is defined by the appended claims.

Application of the invention is suitable for age synchronization of insect embryos, typically BSF embryos, and controlling hatching of eggs, e.g. BSF eggs, and dosing hatched eggs of e.g. Black soldier fly. More in general, the method of the invention is suitably applied for the age synchronization and dosing of an arthropod. Typically, the method of the invention is used for age synchronization and for dosing of any of the species Lacewings (e.g. Chrysoperla carnea), for which larvae hatched from eggs can be age-synchronized and dosed in appropriate amounts at predetermined and desired time-points to timely get colonies of a desired size at a desired age; Coccinelid beetles (e.g. Cryptolaemus montrouzieri), for which collected eggs can be subjected to the method; predatory bugs (e.g. Macrolophus pygmaeus), for which collected eggs can be subjected to the method and subsequently, nymphs are dosed in appropriate amounts to get accurate numbers in feeding batches to rear until the adult stage or nymphs are provided with the method directly as an end product (flightless nymphs). Of course, the method of the invention can be applied with many other insects, such as pollinators (e.g. the onion fly, Delia antiqua) and predatory beetles (e.g. the greenhouse rove beetle, Dalotia coriaria). Indeed, for terrestrial fly species of which the eggs can be collected, these species are suitably applicable for subjecting eggs to the method of the invention, e.g. for dosing larvae such as age-synchronized larvae or such as larvae that are provided at a predetermined time-point during the day or week. Where appropriate, throughout the specification, and in the claims, the term ‘insects’ can be read as ‘arthropods’, covering for example flies and mites, such as the Black soldier fly, more in particular the eggs, embryos and (neonate) larvae of the Black soldier fly, as well as mites, unless it is clear from the context that specifically insects according to the common definition are referred to. It is appreciated by the skilled person that the method of the invention is suitable for, for example, insect age synchronization and timing of insect age and timing of hatching and controlling insect dose. In particular, the method and means of the invention are applicable and suitable for use in the mass rearing and farming of BSF, and thus in particular for controlling hatching of BSF eggs and neonate larvae.

An aspect of the invention relates to a method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1.

An aspect of the invention relates to a method for controlling hatching of Black soldier fly eggs, comprising the steps of: a) at a time point T1 providing a batch of Black soldier fly eggs for which selected lapsed time period P0 at time point T1 relative to a time point T0 at which first eggs of the batch of eggs are deposited, is known; b) starting at T1, incubating the batch of eggs of step a) for a selected first incubation period P1 ending at a time point T2 relative to T1, at a selected controlled first temperature, bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1; c) starting at T2 of step b), cooling of the batch of eggs of step b) at a selected controlled second temperature lower than the first temperature, for a predetermined second incubation period P4 ending at time point T3, cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2; d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and e) starting at H2, obtaining hatched batch of eggs during a time frame P6. Reference is made to Scheme I and Scheme II, here below.

Throughout the specification, examples, claims, abstract, etc., unless specified differently, when a temperature is referred to, or when a temperature selected from a range of temperatures is referred to, said temperature can either be a discrete value, such as an integer value such as 30° C., 10° C., 14° C., or a non-integer value such as 10.8° C., for which commonly accepted rounding-off applies and for which commonly accepted error margins applies (for example, 30° C. implies 29.5° C. - 30.4° C.), or the temperature can be a range with boundaries indicated by a given temperature range, e.g. 30° C. (±1° C.) or a temperature selected from the range between 0° C. and 13.5° C., or wherein the temperature is within 8° C. - 12° C., or 10° C. ±0.5° C. Thus, unless indicated otherwise, for example the selected controlled first temperature in the method according to the invention can for example be selected as a temperature of 30° C. or 30.0° C., or can be selected as a temperature staying during the indicated time period within a given boundary of for example 28.5° C. and 31° C., or can be any temperature selected from a given range of temperatures throughout the indicated time period, such as the first temperature selected from the temperature range 29.0° C. - 31.5° C., preferably about 30° C.

The batch of BSF eggs is typically provided as one or a multitude such as two - ten ovisites, i.e. (cardboard or polymer) holders for containing insect eggs. Typically, the ovisite is filled with eggs which have a certain defined and selected maximum age difference, relating to the time frame during which the ovisite is exposed to gravid insects such as gravid BSF flies. The ovisites are for example positioned in a breeding cage comprising 1.000 to 50.000 BSF. The ovisites are for example positioned in such a cage comprising gravid BSF for a time frame of between 5 minutes and 24 hours, typically for about 30 minutes to 3 hours, such as about 1 hour, or for about 10-20 hours, such as 12 hours. Eggs collected in such an ovisite have a maximum age difference essentially equal to the time during which the ovisite was exposed to the gravid insects inside the cage. The depositing of eggs inside the cavities and/or holes of an ovisite is for example stimulated by flowing an attractive odor (olfactory attractant) through or near the ovisites, such that gravid insects are attracted to the desired site of depositing eggs. Time point T0 is the moment at which an ovisite is positioned inside a container comprising gravid insects such as gravid BSF, and T0 is therewith essentially the time point at which the first eggs are deposited inside the ovisite. In a harvested, or collected, ovisite, the oldest eggs are thus eggs deposited at T0. During the exposure of ovisites inside e.g. a cage to gravid flies, the temperature and relative humidity are controlled and optimal for e.g. BSF behavior (mating, drinking, depositing eggs, etc.). Typically, the temperature inside the cage comprising the ovisite is between 28° C. and 35° C.; the humidity inside e.g. the cage for gravid BSF flies is an absolute air humidity of between 10 gram H₂O/kg air and 30 gram H₂O/kg air at an air temperature of between 28° C. and 35° C. Typically, the time point T1 is the moment at which an ovisite is collected and removed from a cage comprising gravid BSF flies. Thus, typically, P0 is the time period during which the ovisite was positioned inside a cage comprising gravid flies, which flies deposited their eggs in the ovisite during time period P0 between positioning of the ovisite inside the cage at T0 and harvesting the ovisite filled with eggs for provision of a batch of BSF eggs at time point T1 in step a) of the method of the invention.

Time period P1 is typically shorter than time period P2 according to the invention, in view of desired subsequent incubation steps with the eggs provided in step a) of the invention. If P1 is equal to P2, the incubated eggs kept at the selected controlled first temperature start to hatch at time point H1. Either, neonates can be collected and harvested from time point H1 onwards, directly, or hatching of the eggs can be postponed by starting the cooling step c) of the method of the invention the latest at time point H1, or at a time point beyond T1, the start of the incubation of the eggs at the first temperature. The prerequisite for the start of the cooling step c) of the method of the invention is, that the start time point T2 for the cooling step is selected such that embryos further develop during the cooling step and/or during the subsequent incubation at e.g. the first temperature, again. For BSF embryos, typically, the embryos are older than 1 hr of age when the cooling step c) starts at T2, preferably, older than 2-5 hr of age. At the same time, for BSF embryos, typically, the embryos are younger than 33 hr of age or older than 36 hr of age, at the start of the cooling step c) at T2, when the BSF eggs are incubated at 30° C. and 80% RH, since T1 and up to T2.

According to step bb) of the method of the invention, incubation of Black soldier fly eggs at the selected controlled first temperature allows Black soldier fly eggs to hatch from a time-point H1 onwards relative to the time point T0, during a time frame P2 (typically less than 24 hr when the hatching eggs have an age difference of 12 hr or shorter such as 12 hr or 1 hr), when Black soldier fly eggs are incubated at the first temperature (typically 28° C. - 33° C., such as about 30° C., at a relative humidity of 70%-90%, preferably about 80%) for a time period P3 starting at T0 and ending at H1, typically about 58-60 hr relative to the age of the oldest embryos (thus: if an ovisite comprises eggs with an age difference of e.g. 1 hr or 12 hr, P3 ends 57-59 hr after harvesting the ovisite from a cage, or 46-48 hr after harvesting the ovisite from a cage, respectively). The time-point H1 is typically about 58-60 hours later compared to T0 at which the oldest BSF eggs are deposited, wherein all collected eggs in a batch of BSF eggs have a known maximum age difference equal to the time the ovisite was positioned inside a cage comprising gravid BSF female flies.

Given T0 for a certain batch of BSF eggs, at known and predetermined incubation temperature and RH, the length of P3 until hatching of the eggs starts at time point H1, can be established. For example, H1 can be determined by visual inspection (neonate larvae start emerging from the eggs in the ovisite and are for example collected on a scale, or visualized such as by a (high speed) camera, for example a camera suitable for imaging and counting neonates. A method for determining H1 is for example outlined in the examples section, Example 1. Similarly, the length of P2, the time that neonates emerge from the eggs contained in an ovisite, can be determined by counting emerging larvae, by weighing the mass difference between the ovisite before time point H1 and at time points during P2: if no further larvae emerging from the ovisite are for example counted, hatching is completed, and/or when the weight of the ovisite does not decline anymore, hatching is completed. It is part of the invention that time period P2 for hatching of BSF eggs starting from time point H1 is for some embodiments defined as the time period P2 wherein at least 75% of the maximum number of neonates or at least 75% of the maximum weight of neonates, emerged from the batch of eggs. Similarly, it is part of the invention that time period P6 for hatching of BSF eggs starting from time point H2 is for some embodiments defined as the time period P6 wherein at least 75% of the maximum number of neonates or at least 75% of the maximum weight of neonates, emerged from the batch of eggs. Typically, P2 may last for 14 hours or longer, such as 14 hr - 24 hr, for example 16 hr - 20 hr. Typically, P6 is as long as P2 for some embodiments, and shorter for other embodiments. P6 can be 2 hr - 6 hr shorter than P2, depending on the time point T2 at which the cooling step c) of the method of the invention relative to time point T0 and T1. That is to say, when T2 is 5 hr to 0 hr before H1, typically 2 hr to 0 hr before H1, the cooling step c) results in a short time frame P6 for hatching of eggs to an extent of 75% or more relative to the maximum number of hatching eggs, compared to duration of P2 for embryos which were incubated steadily at a constant first temperature of e.g. 30° C.

It will be appreciated by the skilled person that the method of the invention is also suitable for controlling hatching of insect species different from BSF. For such species, similar to BSF, the particular first temperature is known or is established, and P3 and H1 are established or known. In general, establishing such a first temperature, P3 and H1 can be performed for any insect embryos, for which the age of the eggs is known. Example 1 is a typical example of establishing the P3 and H1 for BSF embryos, for which a first temperature is known, i.e. here 30° C.

The prerequisite for the selected time point T2 at which the cooling step c) of the method of the invention starts, is fulfilled if the following requirements of step cc) of the method of the invention are met: the time point T2, relative to the age of the oldest eggs in the batch of eggs provided in step a) of the method, is selected such that cooling of Black soldier fly eggs at the second temperature (e.g. a temperature of between 3° C. and 12° C.) allows Black soldier fly eggs to hatch from a time-point H2 onwards relative to the time point T0, when Black soldier fly eggs are first incubated at the controlled selected first temperature (typically 26° C. - 34° C., such as 29° C. - 31° C., for example about 30° C.) for the time period P1 starting at T1 and ending at T2 according to step b) of the method of the invention, and subsequently incubated at the second temperature (which is lower than the first temperature; e.g. 2° C. -11° C.) for the second incubation period P4 starting at T2 and ending at T3, followed by immediate warming the cooled Black soldier fly eggs at the selected controlled first temperature of step b) of the method of the invention for a third incubation period P5 starting at T3 and ending at time point H2 at which the warmed/cooled/again warmed eggs begin hatching. Thus, selecting any time point T2 as the starting point for incubating the embryos of BSF at a lower temperature than the first temperature, i.e. the second temperature, that would result in hampering or preventing the subsequent hatching of the eggs, once warmed again at the e.g. first temperature, is not part of the invention. Only those time points T2 at which the cooling step c) of the method of the invention can start, resulting in hatching eggs once the cooled eggs are again brought back to the warmer first temperature and incubated at this first temperature for a certain time frame, are part of the invention. Determining suitable time points T2 is for example established with the use of imaging techniques and/or larvae counting techniques and/or BSF egg weighing techniques, such as described in the examples section. For example, a series of e.g. 21 ovisites comprising BSF eggs is provided for which T0 is known. An ovisite is incubated continuously, starting at T1, at a constant temperature of for example 30° C. (and 80% RH, in the dark), and the extent of hatching of the eggs within 24 hr starting from the time point at which first neonates emerge from the eggs, is measured as the weight difference of the ovisite comprising all developing embryos and the ovisite at the end of the 24 hr period of hatching eggs. The hatching is also monitored qualitatively by visual inspection of the hatching eggs (larvae are visible below the ovisites positioned above a horizontal surface). The result provides a reference value for hatching success rate, set to 100% (control). The further 20 ovisites are also incubated at e.g. 30° C. (and 80% RH, in the dark), starting at T1, and at each time point at which a further 3 hr incubation time lapses, an ovisite is cooled to e.g. 10° C. (and 80% RH, in the dark) and kept at this lower temperature for e.g. 24 hr, and subsequently warmed back to e.g. 30° C. (and 80% RH, in the dark), and the time point at which hatching starts is established, as well as the extent of hatching during 24 h from the start of hatching. For example, within the time window 0 hr - 57 hr, 20 ovisites are treated this way, wherein the start of the cooling period differs 3 hr between each two subsequent ovisites subjected to the cooling step. All time points at which cooling started and which were not detrimental to hatching of eggs, once ovisites were warmed back to the first temperature, are suitable time points T2 for starting the cooling of step c) of the method of the invention.

Preferred is the method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
   bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3, wherein the BSF embryos are older than 2-5 hr of age when the cooling step c) starts at T2, and the embryos are younger than 33 hr of age or older than 36 hr of age, at the start of the cooling step c) at T2, when the BSF eggs are incubated at 30° C. and 80% relative humidity (RH), since T1 and up to T2,
   cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6.Preferred is the method of the invention, wherein relative humidity during any one or more of P0, P1, P2, P3, P4, P5 and P6, preferably during all of P0, P1, P2, P3, P4, P5 and P6 is controlled and is kept at a predetermined value.

Preferred is the method of the invention, wherein relative humidity during any one or more of P0, P1, P2, P3, P4, P5 and P6, preferably during all of P0, P1, P2, P3, P4, P5 and P6, is controlled and is kept at a predetermined value, wherein the predetermined value for the relative humidity is 65% -100%, preferably 70% - 95%, more preferably 75% - 90%, most preferably 77% - 85%, such as 80% relative humidity, or 78% - 82%. Controlling RH contributes to the success of hatching of the eggs, i.e. the number of successfully developing embryos and eventually hatching eggs. RH of 80% when the controlled selected first temperature and/or second temperature are about 30° C. and between 5° C. and 10° C., respectively, for example, results in a number of hatched eggs nearing 100% compared to control, when the step c) of the method of the invention (start of cooling period) is at least about 35 hr post depositing of the BSF eggs, such as 38 hr-44 hr since the oldest eggs are deposited (and the youngest eggs are at least 35 hr of age). During the time course of embryo development when eggs are incubated at the first temperature, e.g. 30° C., the eggs may lose about up to 25% weight likely due to evaporation of water (drying of the eggs). Fortunately, the extent of hatching still can reach near 100% compared to control, showing that at least 75% of the eggs hatch compared to control, e.g. on a weight basis.

Throughout the specification, examples, claims, abstract, etc., unless specified differently, when a relative humidity (RH) is referred to, or when a RH selected from a range of RH is referred to, said RH can either be a discrete value, such as an integer value such as 65%, 75%, 80%, or a non-integer value such as 78.5%, for which commonly accepted rounding-off applies and for which commonly accepted error margins applies (for example, 80% implies 79.5% - 80.4%), or the RH can be a range with boundaries indicated by a given RH range, e.g. 73% (±7%) or a RH selected from the range between 70% and 99.9%, or wherein the RH is within 68% - 98%, or 80% ±4%. Thus, unless indicated otherwise, for example the relative humidity during any one or more of P0, P1, P2, P3, P4, P5 and P6 in the method according to the invention can for example be selected as a RH of 80% or 86%, or can be selected as a RH staying during the indicated time period within a given boundary of for example 70% and 90%, or can be any RH selected from a given range of RH throughout the indicated time period, such as RH selected from the RH range 75% - 90%, preferably 80%.

Preferred is the method of the invention, wherein time period P0 which lapsed at time point T1 relative to the time point T0 at which the first eggs of the batch of eggs are deposited, is at least 90 minutes and at longest the duration of time period P3, wherein P3 has the length of the time period starting at time point T0 and ending at time point H1 at which Black soldier fly eggs start hatching after incubation at the first temperature, according to step bb). For BSF embryos, the inventors established that when eggs are first incubated at the first temperature for at least 90 minutes, followed by the cooling period of step c) of the invention, embryos will develop and eggs will ultimately hatch when further incubated at the first temperature after the cooling period at the second temperature. Starting the cooling step c) before the eggs are incubated for 90 minutes at the first temperature such as about 30° C. results in hardly any hatching BSF eggs.

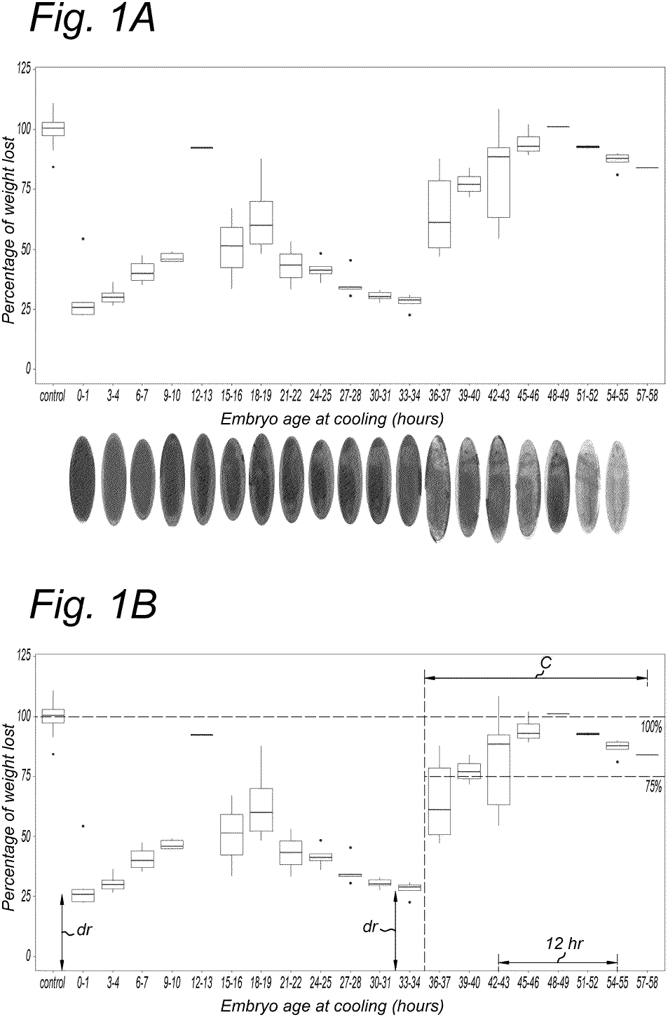

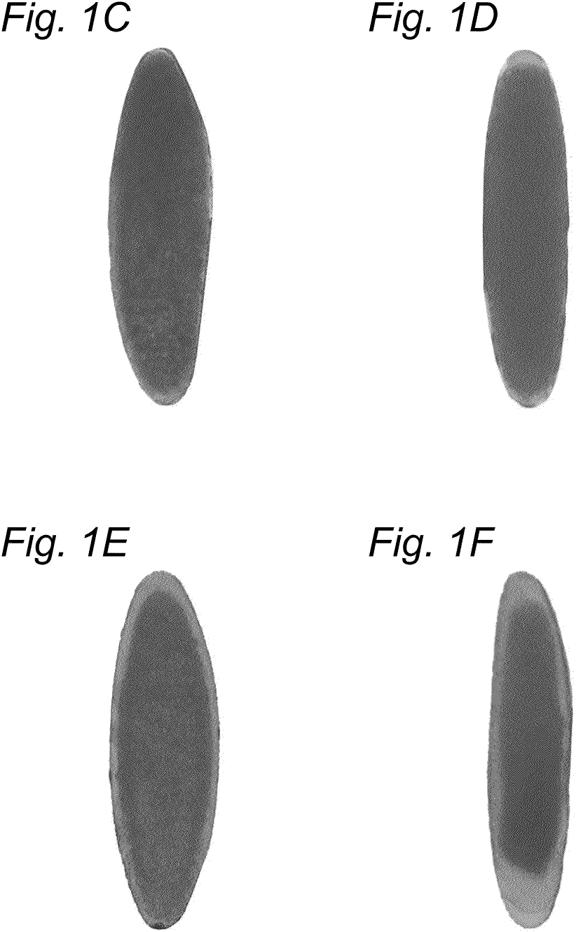

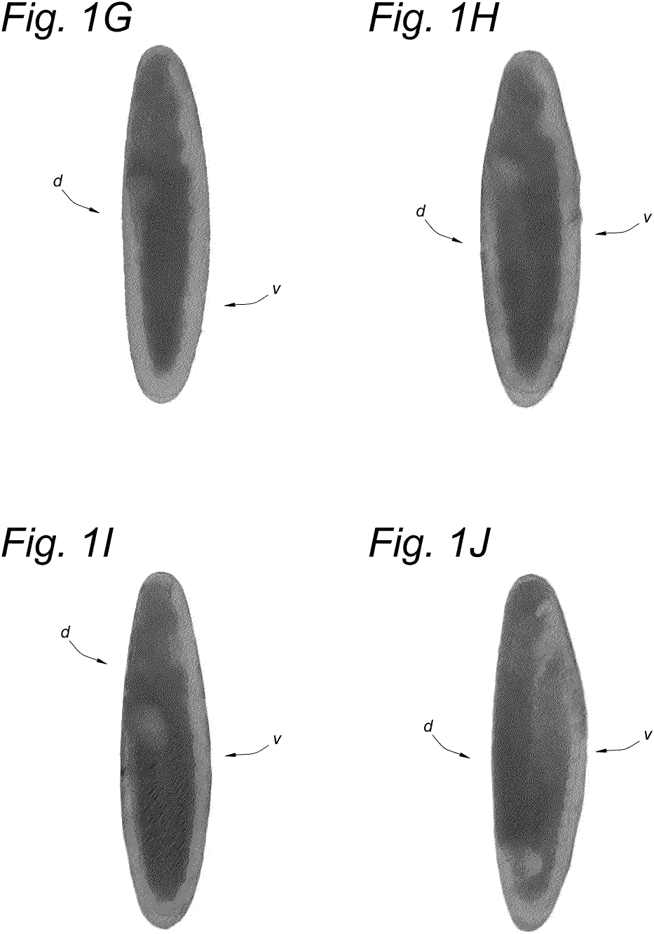

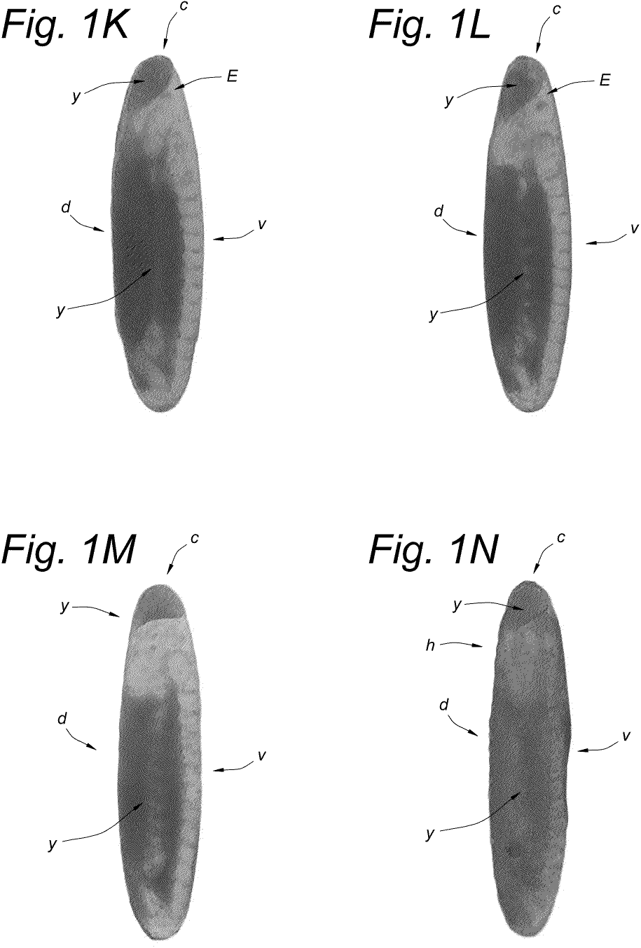

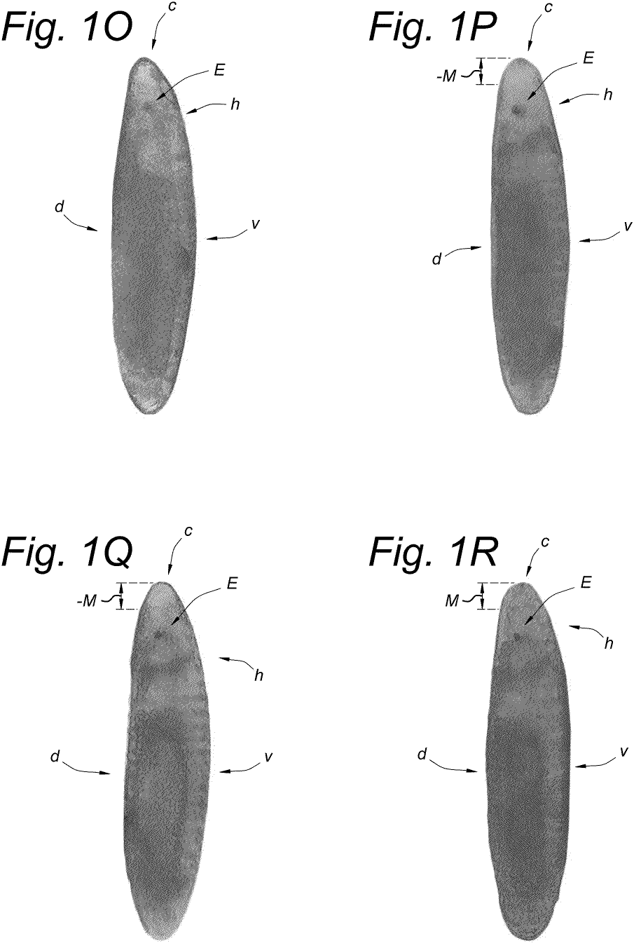

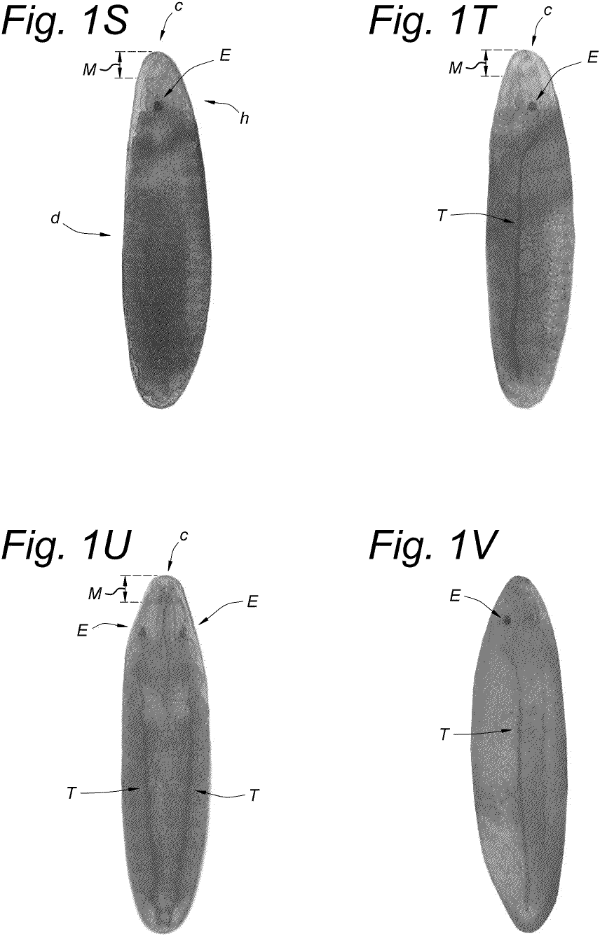

Preferred is the method of the invention, wherein time period P0 which lapsed at time point T1 relative to the time point T0 at which the first eggs of the batch of eggs are deposited, is at least 90 minutes and at longest 33.5 hours, preferably between 3 hours and 31 hours, more preferably between 6 hours and 28 hours, most preferably between 7 hours and 25 hours, such as between 8 hours and 24 hours. The inventors established that in particular, at least a fraction of embryos develop normally when the cooling step c) starts after at least about 90 minutes incubation of the eggs at the first temperature, and starts before about 33.5 hours lapsed since the eggs were deposited, when the eggs are incubated at 30° C. When BSF eggs are incubated at 30° C. and 80% RH, starting the cooling phase in step c) of the method of the invention at a time point between about 33 hr - 34 hr since T0 reduces strongly the number of hatching eggs. Surprisingly, the hatching rate dramatically increases to near 100% when the cooling step c) starts at a time point T2 beyond 34 hr since T0 for BSF. Example 1 and FIG. 1A illustrate this surprising finding of the invention. When the BSF eggs are first incubated for between 47 hr and for example 54 hr at the first temperature, subsequently cooled down to the second temperature, for example about 10° C., and again incubated at the first temperature (e.g. 30° C.), about 100% of the BSF eggs can hatch compared to control. According to the invention the eggs are kept at the second, lower, temperature for at least 1 minute and up to 3 weeks, for example for 2 hr, 6 hr, 24 hr, 48 hr, 72 hr, 120 hr. Since embryos further develop once incubated at the first temperature after the cooling step, and since the eggs hatch similarly to control eggs which were not subjected to a cooling step, the method of the invention allows for selecting and controlling the absolute time point and the time frame at which and in which e.g. BSF eggs hatch. If hatching BSF eggs are required or desired at a certain time point beyond e.g. about 58 hr - 60 hr since a certain time point T0 at which the eggs are deposited, the method of the invention allows selecting a suitable time point T2 for starting the cooling step c) of the method of the invention, and selecting the duration of the second incubation period P4 at the second temperature, from the broad time window between about 1 minute and e.g. 4 weeks, typically between a few hours (1, 2, 3, 4, 5, 6, 7, 8,9, 10, 11, or 12 hr) and a few days (¾ day, 1 day, 1.5 days, 2 days, 2.5 days, 3 days, 4 days, 5 days, 6 days, 7 days, 10 days, 12 days, 2 weeks, 3 weeks, 1 month), therewith effectively delaying the time point at which the BSF eggs start hatching with at least the cooling period, i.e. the second incubation period P4. The method of the invention provides flexibility forthe mass rearing and mass farming of e.g. BSF. Delivery of selected numbers or grams of neonates at a certain moment in time, and/or at a selected time interval for example during a certain time frame of the day, week, month, year, has now become possible due to the invention. For example, a series of ovisites comprising BSF eggs of the same or similar age can be divided in groups of ovisites, wherein each group of ovisites is kept at cool temperature starting at a different T2 and/or for a different second incubation period P4, when for example T2 is the same for each group of ovisites. As a result, from the same batch of BSF eggs (e.g. consisting of multiple ovisites), during a selected and predetermined time window, hatching eggs can be provided starting from a selected and predetermined moment in time, starting at least about 58 hr - 60 hr after the first eggs in the (first ovisite(s) of the) batch were deposited. Alternatively or additively, deposited (BSF) eggs which are deposited at different absolute time points, can hatch starting from the same or similar time point H2 by applying the method of the invention: the eggs that are deposited earlier than subsequently deposited eggs, are kept at the second temperature for a longer timer period P4, until the embryo development of eggs is (near) synchronized. This way, separate ovisites comprising eggs with an absolute age difference of for example 1 day, 2 days, 3 days, 1 week, etc., can hatch at about the same absolute time point H2, due to selected time windows P4 with different lengths.

Preferred is the method of the invention, wherein time period P0 which lapsed at time point T1 relative to the time point T0 at which the first eggs of the batch of eggs are deposited, is at least 34.5 hours and at longest 60 hours, preferably between 35 hours and 59 hours, more preferably between 36 hours and 58 hours, most preferably between 38 hours and 57 hours or between 38 hours and 58 hours or between 38 hours and 56 hours or between 40 hours and 56 hours or between 42 hours and 55 hours or between 43 hours and 54 hours, such as 42 - 56 hours, 45 - 58 hours, 45 - 57 hours or 45 - 56 hours. To the surprise of the inventors, starting the cooling phase P4 at a time point of about 34.5 hr or later since depositing of the eggs results in the highest hatching success, compared to eggs that were cooled at a T2 between 3 hr and 31 hr post-depositing of the eggs. FIG. 1A illustrates the difference in hatch rate or success compared to control eggs which were not subjected to a cooling step. Starting cooling of embryos after lapse of at least 36 hr post depositing of the eggs results in near 100% hatch success for the time window up to 58 hr. Imaging of the embryos at established time points since depositing of the eggs and subsequent incubation at the first temperature (here, 30° C., 80% RH), revealed that dorsal closure appears to be contributing to the success of hatching after a cooling period starting at a T2 beyond 36 hr, without wishing to be bound by theory. Similarly, starting the cooling step c) of the method of the invention shortly after consumption / migration of yolk (away) from the cranial side of the embryo, at about 36 hr post-depositing of the eggs, appears to be beneficially contributing to the relatively high extent of hatching eggs, without wishing to be bound by theory. Further reference is made to the Example 1 and FIGS. 1B-1V.

Preferred is the method of the invention, wherein the time period P3 of step b) is between 58 hours and 62 hours. The inventors established that for BSF eggs, hatching starts at about 60 hr post depositing of the eggs, when the eggs are incubated at about 30° C. and 80% RH. The skilled person will appreciate that according to the method of the invention, P3 can be adjusted and selected by adjusting the first temperature within the boundaries for facilitating development of the embryos. For example, for BSF, the first temperature can be selected from the temperature range between for example about 18° C. and 34° C., although a temperature in the range 26° C. - 32° C. is preferred. A RH of at least 65% is preferred, such as 73% - 93%.

Preferred is the method of the invention, wherein the time frame P2 of step b) is between 12 hours and 20 hours, preferably between 14 hours and 18 hours starting from H1. Typically, for a first temperature of about 30° C. and at 80% RH, embryo development takes about 58-62 hours, and subsequent hatching of a batch of eggs which have a maximum age difference based on the moment of depositing of the eggs, of about 12 hours, typically at least about 75% of the eggs hatch within 20 hours, or within 18 hours.

Preferred is the method of the invention, wherein the time frame P6 is shorter than the time frame P2, preferably P6 is shorter than 14 hours, more preferably between 3 hours and 11 hours, most preferably between 4 hours and 10 hours such as 4-6 hours. The inventors established that P6 is shorter than P2 when the cooling step c) of the method of the invention is starting at time point T2 selected within about 5 hours before hatching starts, e.g. 0-1 hr before hatching starts, wherein P4 is about 1 hr - 12 hr, such as about 2 hr - 6 hr, before the temperature is increased from the second temperature, for example 5° C. or 10° C., back to the first temperature, for example 29° C. - 31° C., e.g. 30° C. Selecting time point T2 close to the moment H1 at which hatching of warmed / cooled / again warmed eggs start hatching, e.g. 2 hr before hatching or 6 hr before hatching, results in a shorter time period before eggs are hatched, for example before at least 75% of the eggs are hatched, compared to a time point T2 which is selected 10 hr to 20 hr before eggs would start hatching if no cooling period P4 would be applied. Thus, selecting T2 close to H1 effectively compressed the time window P6 in which at least 75% of the eggs hatch. For example, starting the cooling period P4 at 2 hr before H1 or at 6 hr before H1 shortened the hatching period P6 with at least 2 hours compared to the hatching period P2 for eggs which are not subjected to a cooling step c) according to the method. The invention provides a method for controlling and selecting the time of hatching of (BSF) eggs, and provides a method for controlling and selecting the time window in which a selected batch of BSF eggs hatches, wherein the time window is between P2 and P6. A shorter time window in which neonates emerge from the eggs is for example beneficial for efficient use of machinery and equipment: more batches of eggs can subsequently hatch in the same climate room when the time for hatching of a certain batch of eggs is shortened compared to control (no cooling step applied).

Preferred is the method of the invention, wherein the predetermined second incubation period P4 is between 1 minute and 21 days, preferably between 2 minutes and 14 days, more preferably between 5 minutes and 10 days, most preferably between 10 minutes and 7 days. The method of the invention provides a flexible way of providing hatching eggs at a desired moment in time. If during for example a day of operation at a farm for mass farming e.g. BSF, a pause of e.g. 1 or a few hours is required for maintenance of machinery, the method provides the possibility to interrupt the supply of hatching eggs by subjecting embryos to step c) of the method of the invention, wherein P4 spans the desired pause time. For example, ovisites comprising deposited eggs which will start hatching at a weekend day when incubation at the first temperature would be continued (in case of non-24 hr operation of a BSF factory), can be kept at the second temperature over the weekend for the time period P4 as selected and desired (weekend long), and can be further incubated at the first temperature, once farm logistics allows, e.g. at Monday.

Preferred is the method of the invention, wherein the predetermined second incubation period P4 is between 30 minutes and 6 days, preferably between 1 hour and 5 days, more preferably between 2 hours and 3 days, most preferably between 5 hours and 2 days, such as between 6 hours and 12 hours. Keeping embryos at the second temperature for P4 of hours to days allows for example for transfer of eggs within a factory or farm under controlled and cooled conditions, delaying further development of the embryos when required, and allows for transport of embryos for a longer period of time compared to when step c) of the method of the invention would not be applied. By applying the cooling period P4 of the invention, the moment the (BSF) eggs start to hatch is effectively delayed with at least the time period P4, when the eggs are warmed again to the first temperature. Typically, the first temperature is for BSF about 27° C. - 33° C., and the second temperature is typically between 1° C. and below 13.5° C., preferably 7° C. - 11° C. RH is typically 70% - 85%.

Preferred is the method of the invention, wherein the predetermined second incubation period P4 is between 1 hour and 4 days, preferably between 2 hours and 3 days, more preferably between 6 hours and 2 days, most preferably between 16 hours and 1 day. Selecting P4 within these ranges allows for optimal timing of hatching during a labor day at e.g. a mass rearing farm for e.g. BSF.

Preferred is the method of the invention, wherein the selected controlled second temperature is between 0° C. and 17° C., preferably 1° C. - 16° C., more preferably 2° C. - 15° C., most preferably, 3° C. - 14° C.

Preferred is the method of the invention, wherein the selected controlled second temperature is between 4° C. and below 14° C., preferably 5° C. - 13° C., more preferably 6° C. - 12° C., most preferably 7° C. - 11° C., such as 5° C. - 12° C. or 8° C. - 10° C. Preferred is the method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
   bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3,
   cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6, wherein the selected controlled second temperature is between 4° C. and below 14° C., preferably 5° C. -10° C.

The inventors established for BSF eggs which are incubated starting at T1 at 30° C. (80% RH), that embryo development is (near) halted when the second temperature is lower than 14° C. (and 0° C. or higher), based on imaging of embryos shortly before and after the start and end of second incubation period P4. The eggs incubated at any of these temperatures between 0° C. and below 14° C. required subsequent incubation at the first temperature after the cooling step, before hatching of the eggs was established. In contrast, when at T2 eggs are cooled to a controlled selected second temperature of 14° C. - 16° C. for the second incubation period P4, embryo development appears not to be stopped during P4, though appears to be slowed down, since incubation period P5 is shorter for eggs incubated at these temperatures compared to period P5 up to the start of hatching at time point H2 for eggs which were incubated during P4 at lower temperature (e.g. at 5° C., 10° C. or 12° C.). Selecting the second temperature from the range between 0° C. and e.g. 16° C. thus further allows for determining and selecting the start of the hatching BSF eggs. By selecting the temperature for the period P4, the length of the subsequent period P5 is influenced, and therewith the start of the hatching. This provides further flexibility for the insect farmer in need of timing the moment of hatching and in need of the synchronization of hatching of separate batches of (BSF) eggs, harvested for example at different moments in time, e.g. from different cages, e.g. at different days.

Preferred is the method of the invention, wherein the selected first incubation period P1 is starting at time point T1, wherein T1 is selected from a time point between 1 hour relative to time point T0 at which the first eggs of the batch of eggs are deposited and time-point H1 at which Black soldier fly eggs start to hatch, according to step bb), preferably T1 is selected from a time point between 2 hours and 60 hours, more preferably between 3 hours and 58 hours, most preferably between 4 hours and 57 hours. As said, it is beneficial if eggs are first incubated at the controlled selected first temperature for at least an hour such as about 2-3 hours, before the eggs are subjected to the cooling step c) of the method of the invention, when the extent of the hatching of eggs is considered. Most beneficially, the step c) starts at a time point T2 which is at least about 36 hr post depositing of the eggs, when the eggs are for example BSF eggs, incubated at a first temperature of about 30° C. The number of hatching eggs within a defined time frame P6 is optimal if T2 is about 40 hr - 60 hr post-egg depositing, such as about 44 hr - 56 hr after the oldest eggs were deposited.

Preferred is the method of the invention, wherein the selected first incubation period P1 is starting at time point T1, wherein T1 is selected from a time point between 3 hours relative to time point T0 at which the first eggs of the batch of eggs are deposited and 32 hours, preferably T1 is selected from a time point between 4 hours and 31 hours, or is between 35 hours and 60 hours, preferably between 36 hours and 58 hours, more preferably between 37 hours and 57 hours.

Preferred is the method of the invention, wherein the selected controlled first temperature is between 25° C. and 35° C., preferably between 26° C. and 34° C., more preferably between 27° C. and 33° C., most preferably between 28° C. and 32° C., such as 28.5° C. - 30° C. or 30° C. - 31.5° C. Embryos of e.g. BSF develop properly when incubated at a temperature selected from these ranges. Typically efficient embryo development is established when BSF eggs are incubated at 30° C. (80% RH).

Preferred is the method of the invention, wherein the selected first incubation period P1 and the third incubation period P5 together are the same or longer than time period P3, preferably between 1 hour and 10 hours longer, more preferably 2 hours - 6 hours, most preferably 3 hours - 4 hours. Preferred is the method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
   bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3,
   cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6, wherein the selected first incubation period P1 and the third incubation period P5 together are the same or longer than time period P3, preferably between 1 hour and 10 hours longer, more preferably 2 hours - 6 hours, most preferably 3 hours - 4 hours.

The inventors established that the total incubation time at the first temperature (e.g. about 30° C.) for BSF embryos can be prolonged with for example 1 hr - 2 hr when the eggs are cooled to the second temperature starting from a time point T2 about at least 36 hr post depositing. The application of the method of the invention provides an opportunity to delay the embryo development with the duration of the second incubation period P4, and with an additional time window of the indicated few hours, therewith providing the mass insect farmer with the possibility to control the time point at which hatching eggs are needed.

Preferred is the method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
   bb) wherein incubation of Black soldier fly eggs at the first temperature allows Black soldier fly (BSF) eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3, wherein the BSF embryos are older than 2-5 hr of age when the cooling step c) starts at T2, and the embryos are younger than 33 hr of age or older than 36 hr of age, at the start of the cooling step c) at T2, when the BSF eggs are incubated at 30° C. and 80% relative humidity (RH), since T1 and up to T2,
   cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6, wherein the selected first incubation period P1 and the third incubation period P5 together are the same or longer than time period P3, preferably between 1 hour and 10 hours longer, more preferably 2 hours - 6 hours, most preferably 3 hours - 4 hours.

Preferred is the method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
   bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3,
   cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6, wherein the selected controlled second temperature is between 4° C. and below 14° C., preferably 5° C. -10° C., and wherein the selected first incubation period P1 and the third incubation period P5 together are the same or longer than time period P3, preferably between 1 hour and 10 hours longer, more preferably 2 hours - 6 hours, most preferably 3 hours - 4 hours.

Preferred is the method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
   bb) wherein incubation of Black soldier fly eggs at the first temperature allows Black soldier fly (BSF) eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3, wherein the BSF embryos are older than 2-5 hr of age when the cooling step c) starts at T2, and the embryos are younger than 33 hr of age or older than 36 hr of age, at the start of the cooling step c) at T2, when the BSF eggs are incubated at 30° C. and 80% relative humidity (RH), since T1 and up to T2,
   cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6, wherein the selected first incubation period P1 and the third incubation period P5 together are the same or longer than time period P3, preferably between 1 hour and 10 hours longer, more preferably 2 hours - 6 hours, most preferably 3 hours - 4 hours, wherein the selected controlled second temperature is between 4° C. and below 14° C., preferably 5° C. - 10° C.

Preferred is the method of the invention, wherein during time frame P2 and/or during time frame P6, the temperature during hatching of the eggs is between 25° C. and 35° C., preferably between 26° C. and 34° C., more preferably between 27° C. and 33° C., most preferably between 28° C. and 32° C., such as 28.5° C. - 30° C. or 30° C. - 31.5° C.

Preferred is the method of the invention, wherein the batch of eggs provided in step a) comprises between 1 gram eggs and 3.000 kg eggs, preferably 5 gram - 2.000 kg, more preferably 20 gram -1.000 kg, most preferably 100 gram - 500 kg. The method of the invention is applicable for (combined) ovisites comprising less than 1 kg BSF eggs, up to numbers of ovisites applicable for large-scale farming, such as tens to hundreds of ovisites, with combined egg mass of several hundreds to thousands of kilos of BSF eggs, as the demand and requirements for economically mass production of BSF (larvae) may be. The method of the invention can be applied with simple equipment such as a small scale air-conditioned and temperature controlled refrigerator, and can be applied with an industrial scale cooled warehouse with controlled RH, and any size of a temperature-controllable and RH controllable incubation container there in between.

Preferred is the method of the invention, wherein the Black soldier fly eggs of the batch of eggs provided in step a) have a maximum age difference of between 30 minutes and 18 hours, preferably 1 hour - 16 hours, more preferably 2 hours - 14 hours, most preferably 3 hours - 12 hours, wherein T1, T2, T3, H1 and H2 are relative to the time point T0 at which the first eggs of the batch of eggs are deposited. Typically, the age difference between eggs in a single ovisite harvested during routine farming at an industrial scale BSF farm or factory, is between 0 hr and 16 hr, or 0 hr - 1 hr, or 0 hr - 12 hr, which translates to ovisites being exposed to gravid female BSF which could deposit their eggs for 16 hr, 1 hr or 12 hr, respectively, before ovisites are removed from e.g. a mass-rearing BSF cage. The age difference of eggs in a single batch of BSF eggs can be selected from a wide range of ages according to the method of the invention, although an age difference of 10 hr - 16 hr is beneficial when maximum hatch rate is desired. That is to say, when eggs in a batch of BSF eggs have a maximum age difference of for example 12 hr, the second incubation period at for example 10° C. can beneficially start when the eggs are e.g. 38 hr - 50 hr of age, or 44 hr - 56 hr of age. The age range at which the cooling step c) of the method of the invention can start, which provides highest hatch rates (see for example FIG. 1B), is about 39 hr - 58 hr for BSF embryos post depositing of the eggs.

Preferred is the method of the invention, wherein the time point H1 of step bb) relative to time point T0 is 63 hours or shorter, preferably 56 hours - 62 hours, more preferably 57 hours - 61 hours, most preferably 58 hours - 60 hours. The inventors established for BSF embryos that an incubation of about 60 hr is sufficient to induce hatching, when the first temperature is about 27° C. - 32° C. such as about 30° C., and RH is about 73% - 95%, such as 80%. A relatively lower first temperature, such as 27° C. results in H1 being relatively later in time relative to T0, whereas a relatively higher first temperature may shorten the time period before H1 is reached relative to T0, such that selecting the first temperature provides the opportunity to further control and select the moment BSF eggs start hatching.

Preferred is the method of the invention, wherein time point T2 for starting cooling of the batch of eggs is at least 2 hours after time point T0 at which the first eggs of the batch of eggs are deposited, and at least 3 hours before yolk in the eggs is absent in the cranial side of embryos in the eggs or at least 3 hours after yolk in the eggs is absent in the cranial side of the embryos in the eggs. The inventors established that the extent of hatching eggs is higher when the time point T2 is selected at least 2 hr -4 hr after depositing of the eggs, and at the same time about 3 hr before yolk becomes absent in the cranial side of the embryo or about 3 hr after yolk was still present at the cranial side of the embryo. Lower extent of hatching eggs was established when T2 was selected closer to T0, or when T2 was selected at a time point at which yolk at the cranial side would become absent within 3 hr of selected T2, or when T2 was selected at a time point at which yolk was not yet not present anymore for at least 3 hr.

Preferred is the method of the invention, wherein time point T2 for starting cooling of the batch of eggs is at least 2 hours after time point T0 at which the batch of eggs is deposited, and at least 2 hours before dorsal closure in embryos in the eggs or at least 1 hour after dorsal closure in the embryos in the eggs, preferably 2 hours - 32 hours before dorsal closure in the embryos in the eggs, more preferably 3 hours - 20 hours before dorsal closure in the embryos in the eggs or 1 hour - 25 hours after dorsal closure in the embryos in the eggs, most preferably 3 hours - 22 hours after dorsal closure in the embryos in the eggs. The inventors established that the extent of hatching eggs is higher when the time point T2 is selected at least 2 hr - 4 hr after depositing of the eggs, and at the same time at least about 2 hr or more before dorsal closure in the embryos in the eggs or at least about 1 hr or longer after dorsal closure in the embryos in the eggs. Lower extent of hatching eggs was established when T2 was selected closer to T0, or when T2 was selected at a time point at which dorsal closure would become established within 2 hr of selected T2, or when T2 was selected at a time point at which dorsal closure was not yet established for at least 1 hr.

Preferred is the method of the invention, wherein the eggs comprising the embryos and the yolk and the embryos are visualized using a trinocular microscope provided with a camera, and wherein the eggs, yolk and embryos are magnified at least five times, preferably 5 - 120 times, more preferably 10 - 60 times, most preferably 20 - 40 times, such as 25 times. Imaging embryos inside the BSF eggs is beneficially established with such a trinocular microscope provided with a camera, and the skilled person would appreciate that other means of visualizing and imaging of embryos can equally beneficially be applied for the purpose of determining the presence or absence of yolk at the cranial side of the embryo and/or for establishing the time at which dorsal closure is or is not (yet) established in the embryo.

Preferred is the method of the invention, wherein in step c) T2 is selected from a time point between 25 hours and 0 hour before time point H1 of step bb), preferably between 20 hours and 0 hour, more preferably between 18 hours and 0 hour, most preferably between 16 hours and 0 hour, such as selected from 14 hours - 2 hours or 12 hours - 4 hours. When T2 is selected from these ranges of time points, the extent of hatching of the BSF eggs is higher than when T2 is selected from the time range starting at about 2 hr post depositing of the eggs and about 30 hr post depositing of the eggs. Typically, eggs are incubated at 30° C. before time point T2 is reached.

Preferred is the method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
   bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3,
   cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6, wherein time point T2 for starting cooling of the batch of eggs is at least 2 hours after time point T0 at which the first eggs of the batch of eggs are deposited, and at least 3 hours before yolk in the eggs is absent in the cranial side of embryos in the eggs or at least 3 hours after yolk in the eggs is absent in the cranial side of the embryos in the eggs.

Preferred is the method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
   bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3,
   cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6, wherein time point T2 for starting cooling of the batch of eggs is at least 2 hours after time point T0 at which the batch of eggs is deposited, and at least 2 hours before dorsal closure in embryos in the eggs or at least 1 hour after dorsal closure in the embryos in the eggs, preferably 2 hours - 32 hours before dorsal closure in the embryos in the eggs, more preferably 3 hours - 20 hours before dorsal closure in the embryos in the eggs or 1 hour - 25 hours after dorsal closure in the embryos in the eggs, most preferably 3 hours - 22 hours after dorsal closure in the embryos in the eggs.

Preferred is the method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
   bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3,
   cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6, wherein in step c) T2 is selected from a time point between 25 hours and 0 hour before time point H1 of step bb), preferably between 20 hours and 0 hour, more preferably between 18 hours and 0 hour, most preferably between 16 hours and 0 hour, such as selected from 14 hours - 2 hours or 12 hours - 4 hours.

An aspect of the invention relates to a batch of Black soldier fly eggs comprising embryos and kept at a temperature of between 0° C. and below 14° C. with a relative humidity of 65% or higher, wherein the embryos are capable of developing into Black soldier fly neonates if the temperature is risen to 14° C. - 40° C. while the relative humidity remains 65% or higher.

Preferred is the batch of Black soldier fly eggs comprising embryos according to the invention, wherein the temperature at which said eggs are kept is between 4° C. and lower than 14° C., preferably 8° C. - 12° C., such as 9° C., 10° C. or 11° C., with a relative humidity of 70% - 100%, preferably 75% - 90%, such as about 80%, wherein the embryos are capable of developing into Black soldier fly neonates if the temperature is risen to 16° C. - 35° C., preferably 25° C. - 33° C., more preferably 28° C. - 32° C., such as about 30° C. while the relative humidity remains 70% - 100%, preferably 75% - 90%, such as about 80%.

Preferred is the batch of Black soldier fly eggs comprising embryos according to the invention, wherein the batch of eggs is provided with subsequently step a), step b) and step c) of the method according to the invention.

Preferred is the batch of Black soldier fly eggs comprising embryos according to the invention, wherein the embryos are capable of developing into Black soldier fly neonates if subjected to subsequent steps d) and e) of the method according to the invention.

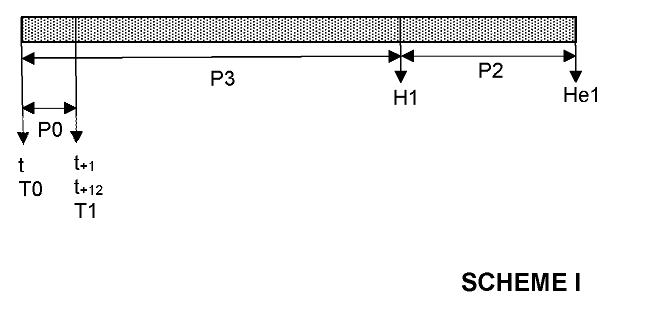

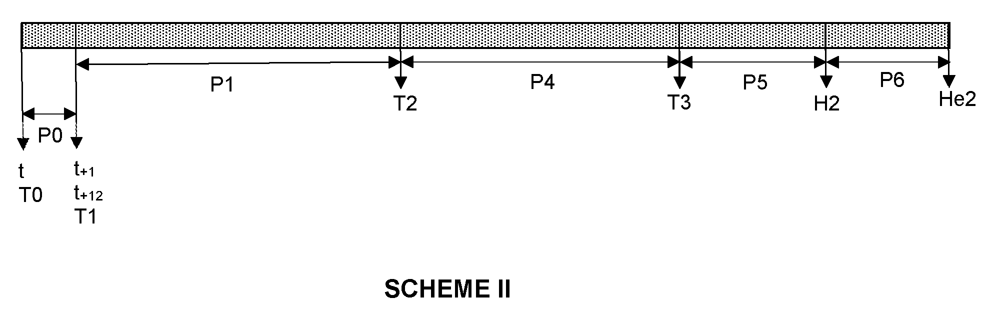

In summary, the invention relates amongst others to a method for controlling hatching of Black soldier fly eggs, comprising the steps of incubating the eggs at a selected temperature for a selected time period; cooling the incubated eggs at a temperature lower than the selected temperature, starting at a selected time point; warming the eggs again at the selected temperature for a further selected period, up till the eggs hatch. Furthermore, the invention relates to a batch of Black soldier fly eggs kept at the temperature at which the eggs are cooled according to the invention, which cooled eggs are capable of hatching once warmed at the selected temperature according to the invention for a predetermined period of time. This way the invention provides for a method for delaying of hatching of an egg. Furthermore, the method of the invention and the batch of cooled eggs of the invention provide a method for synchronizing of hatching of a first egg and a second egg which are deposited at a first time point and at a second time point. In addition, the method of the invention and the batch of cooled eggs of the invention allow for compressing the time window for hatching of a batch of eggs which are deposited within a time frame of e.g. between 0 hours and 12 hours, to a time period of 14 hours or less.

The invention is further illustrated by the following examples, which should not be interpreted as limiting the present invention in any way.

### EXAMPLES

### Example 1

### Pausing of Black Soldier Fly Embryo Development

### Materials and Method

### Egg Collection

Black Soldier Fly eggs were collected in ovisites. For the purpose, empty ovisites were placed for one hour for example 1, or for 12 hours (further examples) in a cage comprising about 10.000 - 30.000 gravid BSF females. Presence of an olfactory attractant in and near the cavities of the ovisites stimulated the gravid flies to deposit the eggs (lay eggs, oviposite) for one hour (example 1) or for 12 hours (further examples) in the cavities of the ovisites. This results in batches of eggs with a maximum age difference of one hour (example 1) or 12 hours (further examples). Throughout the example 1-3, the BSF eggs are therefore referred to as eggs that have an age of t - t₊₁ hours, or t - t₊₁₂ hours, respectively. Gravid female BSF flies start depositing eggs in the ovisites immediately when the ovisites are positioned in the cage, and the flies are still depositing eggs in the ovisites at the moment that the ovisites are removed from the cage (1 hour or 12 hours after the ovisites were positioned inside the cage). This way, the ovisites either comprise eggs which have an age difference of between 0 minutes and 1 hour, when the ovisite was positioned inside the cage for 1 hour, or comprise eggs which have an age difference of between 0 minutes and 12 hours, when the ovisite was positioned inside the cage for 12 hours. Thus, for ovisites which were inside the cage for 1 hour, the age of the eggs is 0 - 1 hour; for ovisites which were inside the cage for 12 hours, the age of the eggs is 0 - 12 hours.

The ovisites comprising the eggs are collected from the cage after one hour or after 12 hours and the ovisites with eggs are stored in the dark, in a climate cabinet (Memmert ICH110; Memmert GmbH + Co. KG, Schwabach, DE) at 30° C. and 80% relative humidity (RH), for indicated time frames.

### Pausing the Development of BSF Embryos by Cooling Collected Eggs

Control ovisites were kept at 30° C. and 80% RH until hatching of the eggs comprised by the ovisites, which started at around 58-60 hours of age of the eggs (57-58 hours continuous uninterrupted incubation at 30° C. and 80% RH). Hatching of the eggs continued for about 18-22 hr.

To achieve pausing of the embryonic development, the eggs have been first incubated at 30° C. and 80% relative humidity for an indicated time span (including no incubation at 30° C. and 80% relative humidity after harvesting the ovisite: direct start of cooling) and have subsequently been placed at a temperature of between 1° C. and 16° C. (i.e. at a temperature of 1, 2, 3, 4, 5, 10, 12, 14 or 16° C.) and 80% RH, for 24 hours. After this cooling period, the eggs are returned to the warmer climate cabinet (30° C. and 80% RH), until these cooled and again warmed eggs were also incubated for 58 hours in total at 30° C. and 80% relative humidity, i.e. for the same incubation time period of 58 hours at 30° C. and 80% relative humidity as the incubation time period of 58 hours for control eggs. Amount of hatching eggs was determined as described hereunder.

The start of the cooling period was at indicated time points since the collection of an ovisite comprising the eggs from the cage, of 0 hours and 3, 6, 9, 12, 15, 18, 21, 24, 27, 30, 33, 36, 39, 42, 45, 48, 51, 54 and 57 hours, such that the eggs had an age at the start of the cooling period of 0-1 hr, 3-4 hr, 6-7 hr, 9-10 hr, 12-13 hr, 15-16 hr, 18-19 hr, 21-22 hr, 24-25 hr, 27-28 hr, 30-31 hr, 33-34 hr, 36-37 hr, 39-40 hr, 42-43 hr, 45-46 hr, 48-49 hr, 51-52 hr, 54-55 hr, and 57-58 hr, respectively. That is to say, eggs that were subjected to a cooling step had a maximum age difference of 1 hour. After the cooling period of 24, 48 or 72 hours (hr), these cooled eggs were warmed up again to 30° C. at 80% relative humidity, and kept at 30° C. for respectively 57 hr, 54 hr, 51 hr, 48 hr, 45 hr, 42 hr, 39 hr, 36 hr, 33 hr, 30 hr, 27 hr, 24 hr, 21 hr, 18 hr, 15 hr, 12 hr, 9 hr, 6 hr, 3 hr, 0 hr, such that the total time of incubation at 30° C. accumulated to 57-58 hr for each tested batch of eggs, which was the same total incubation period at 30° C. as used for the control eggs (continuous warming at 30° C.). After the incubation at 30° C. and 80% relative humidity for 57-58 hr for all test batches of eggs (thus, with the cooling period at the start, or at the end, or at the indicated time points during the incubation period at 30° C. and 80% relative humidity) and control eggs, hatching of the eggs was measured during a period of 24 hr. For each time point for the start of the cooling, a total of six ovisites comprising the eggs, were tested. Each ovisite contained about 2-10 grams of eggs. One gram of eggs yields about 23.000 BSF neonates (first instar larvae) under standard and controlled circumstances developed by the inventors at Protix (Dongen, NL) for testing this current pausing method. The control eggs, which were thus not subjected to a cooling step, consisted of twelve ovisites, all continuously kept at 30° C. and 80% RH for the entire embryonic development period of 57-58 hours, since eggs were deposited (oviposited) and harvested from the cage.

### Imaging Embryonic Development

Embryos were imaged before and after the cooling treatment for assessing whether the embryos continued developing, during the cool period of 24, 48 or 72 hours. For imaging, samples of eggs were taken from the ovisites and mounted onto microscope slides. Imaging was with a Euromex BioBlue.Lab trinocular microscope (Euromex Microscopen BV, Arnhem (NL)) with a Hayear (HY-3609B) camera (Shenzhen Hayear Electronics Co. Ltd., China) mounted on it.

### Assessing Hatching Success of Control Eggs and Cooled Eggs

Weight loss of the ovisites containing the eggs was taken as the measure for hatching success of BSF eggs, for those ovisites which revealed BSF larvae from the eggs (thus, weight loss of ovisites due to other reasons than living neonates emerging from the eggs, is not taken as successful hatching of eggs, since such weight loss of eggs with non-developed embryos or killed embryos is due to for example drying of the eggs). When the hatching success of cooled eggs in the test ovisites, which ovisites thus comprised eggs which were cooled, is comparable to control (hatching success being comparable is set at +/- 25% difference in the weight change when cooled ovisites are compared to control ovisites), it was established that the cooling treatment does not have a negative effect on hatching success rate. The weight loss was recorded using straight bar load cells with 100 grams capacity (3139_0, Phidgets, Inc., Calgary, Canada). Four of these load cells were attached to PhidgetBridge 4-Input (1046_0B, Phidgets, Inc., Calgary, Canada). A change in weight was logged in automated fashion during a time frame of 24 hours, after which essentially no hatching was observed anymore in any of the controls or tests.

### Data Processing

The weight differences data for the series of control and test cooled ovisites have been analysed using RStudio (Open source and enterprise-ready professional software for data science, RStudio Team (2015). RStudio: Integrated Development for R. RStudio, Inc., Boston, MA (USA), www.rstudio.com). We have calculated the mean percentage of weight lost for each treatment.

### Results

The results of BSF egg hatching success for controls and for the test eggs cooled for 24 hours, wherein cooling started at varying time points since eggs were laid, are summarised as boxplots in FIG. 1A and FIG. 1B. The hatching of control eggs, which were not subjected to a cooling step, is set to 100%. The representative state of the embryo at the varying time points at which cooling started, before that start of the cooling period of 24 hr, is shown in FIG. 1A, below the time axis.

With eggs subjected to cooling at time points at which cooling started at between 0-1 hr or 33-34 hr, visual inspection of the phidgets and the ovisites during the 24 hr after the incubation period of 58 h in total at 30° C., revealed that eggs did not hatch at all; no living BSF larvae emerged from the eggs. That is to say, compared to control, starting the cooling of the eggs at any time point between 0-1 hr or 33-34 hr since ovisites containing eggs were harvested from the cage (set as t = 0 hr), results in 100% loss of embryos, without any larvae emerging from the ovisite after a total incubation period at 30° C. for 25-58 hr after the cooling period. The measured weight loss amounting to a minimum of 20-25% weight loss for these eggs which were cooled at the indicated time point since egg harvesting (at 0-1 hr or at 33-34 hr since ovisite harvesting), when none of the cooled eggs hatches, is likely due to dehydration of the eggs (from which no embryos will hatch), and no living larvae were obtained; hatching success was 0%. When cooling started at any time between 3-4 hr post deposition of the eggs in the ovisite and 30-31 hr post deposition of the eggs in the ovisite, embryos developed with a success rate of up to about 50% (FIGS. 1A, 1B). That is to say, living larvae were emerging from the eggs after the cooled eggs were warmed again, though not more than about 25-50% of the maximum number of larvae, obtained with the controls (non-cooled eggs). Since drying of the eggs in which embryos do not develop (normally), accounts for about 25% weight loss of the eggs, it is estimated that when eggs are cooled starting at 3-31 hr after being deposited in the ovisites, at most 25% of the eggs result in viable embryos, wherein the further up to 25% weight loss of the eggs is accounted for by drying out of eggs (dehydration of e.g. non-developing embryos, non-fertilized eggs, dead embryos).

With eggs subjected to cooling at time points at which cooling started at between 36 hr and 58 hr, visual inspection of the phidgets and the ovisites during the 24 hr after the incubation period of 58 h in total at 30° C., revealed that eggs did efficiently hatch (median hatch success varied between about 60% and about 100%, compared to control, which was set to 100%). Hatching success was essentially the same as control (non-cooled eggs) for eggs for which cooling started at 45 hr, 48 hr or 51 hr since harvesting the eggs from the cage. Percentage weight loss in FIGS. 1A and 1B is determined as the decrease of the weight of the ovisite comprising the eggs, compared to the weight of the empty ovisite. A weight loss of the ovisite of 100% corresponds to hatching of eggs into larvae as obtained for the control ovisites. It is assumed that hatching of control eggs reflects maximum achievable hatch success under the selected circumstances: set to 100%.

### Embryo Development

FIGS. 1C-V show photos of the representative embryos of BSF with the indicated age (age difference of 1 hr due to the positioning of the ovisite inside the cage for 1 hr, before harvesting the ovisite comprising eggs). At these indicated ages (time points since ovisite harvest), cooling was started for 24 hr at 10° C. As said, starting the cooling with embryos 0-1 hr or 33-34 hr of age (FIGS. 1A-B, FIG. 1C,

FIG. 1N), does not result in hatched eggs; no living BSF larvae obtained. At the indicated ages of the embryos, cooling to 10° C. apparently hampers any or further development of the embryo and apparently hampers hatching of the egg, such that no fully developed BSF neonates emerge from the eggs.

Starting the cooling with embryos 3-4 hr up to 30-31 hr of age (FIGS. 1A-B, FIGS. 1D-M), does result in hatched eggs to an extent of 25%-50% of the maximum achievable success rate obtained with control, non-cooled eggs (FIG. 1B); living BSF larvae obtained, though less than when no cooling is applied. At the indicated ages of the embryos (3-31 hr post depositing in ovisites and harvest from the cage; eggs are thus first warmed 3-31 hr at 30° C., before the cooling cycle started), cooling to 10° C. apparently allows further development of the embryo when the egg-incubation temperature is risen again to 30° C., and apparently allows hatching of eggs, such that fully developed BSF neonates emerge from the eggs, although less efficiently when compared to control.

The photos of the embryos in FIGS. 1B and 1C-V display the BSF embryonic development in time, for each time point at which a cooling step was started. For the eggs 33-34 hours of age (FIG. 1N), yolk is present at the cranial side of the embryo (indicated with an arrow ‘c’). When the amnioserosa (a cell membrane in the embryo) ruptures, this yolk migrates to the dorsal side of the embryo (indicated with a ‘d’ and an arrow in FIGS. 1N and 1O, in the Figures the dorsal side d is opposing the ventral side v). Yolk y is not present anymore outside the embryo near the head region (cranial, ‘c’) for the embryos at 36-37 hours of age (FIG. 1O), but is now enclosed inside the embryo when embryos are 36 hr of age or older, indicating that the rupture of the amnioserosa has occurred between 33-34 hours of age and 36-37 hours of age for the embryos (indeed, in separate experiments this rupture was observed for embryos 35-36 hours of age). Dorsal closure starts when the retraction of the amnioserosa has finished. From the photos, it was established that dorsal closure has completed when apparent segmentation at the dorsal side of the embryo becomes visible (compare FIGS. 1O and 1P with FIGS. 1Q and 1R). From the photos in FIGS. 1O - 1R it is concluded that dorsal closure has completed in embryos of about 42-43 hours of age. From the photo of FIG. 1O, it is established that the head h of the larvae starts developing rapidly in embryos 36-37 hours of age and older, and the mouthparts M become defined in embryos of 45-46 of age (FIGS. 1R-U; compare with FIGS. 1P and 1Q, wherein the mouth parts are not yet developed (‘-M’)). The trachea T start to become apparent in embryos around 51-52 hours of age (FIG. 1T), which indicates that the larvae are almost ready to emerge (eggs are about to hatch). The photos do not reveal visible changes in embryos of 54-55 hours of age (FIG. 1U) or 57-58 hours of age (FIG. 1V). The eggs start hatching when the embryos have an age of about 58-60 hours.

### Cooling for 24 Hours at 10° C.

Without wishing to be bound by any theory, from the combined data comprising the extent of hatched eggs with cooled embryos compared to controls, and the series of photos taken at time points at which the cooling step was started, the following has become apparent: 1) presence of visible free yolk in the egg appears to prevent or influence successful hatching of eggs after a cooling step; and 2) eggs are hatching after a cooling step (e.g. 24 hr at 10° C.), when the embryos reached a state of development before cooling started, in which the transition from the rupture of the amnioserosa to completion of dorsal closure was established (here, in the current experiment with embryos 36-37 hours of age or older up to 57-58 hours of age (FIGS. 1A, 1B, 1O, 1U, 1V).

Any time point within the period of twelve hours after completion of dorsal closure (BSF embryos 42-55 hours of age, or even up to 58 hr of age, since eggs have been deposited (oviposited) and since collection of the ovisites comprising the eggs, from the cage with gravid BSF females; FIGS. 1Q - 1U), is apparently in particular suitable for starting the cooling step and cool the embryos for 24 hr (or 48 hr or 72 hr or 120 hr) or any time in between. Hatching rate and extent of hatching relative to control, with eggs which were cooled when embryos had the indicated age of 42-55 hours, is comparable to the hatching rate obtainable with control eggs and embryos, not subjected to the cooling step, 80% or higher up to 100%. Thus, a collection of eggs with an age difference of between 0 hr and about 12 hr can be suitably cooled altogether, once the last laid eggs reached an age of 42 hours and the oldest embryos have an age of about 54-55 hr at that time point. This way, all embryos with an age of 42 hours up to embryos with an age of 55 hours will equally efficiently develop resulting in living BSF neonates once the eggs are warmed again, and the respective eggs hatch equally efficient once the eggs are warmed again for about 16 hours. This way, eggs will hatch starting about 3 hours after embryos were warmed again to 30° C. and hatching will last for about 12 hours or longer, with the majority of the eggs being hatched within 12-16 hours.

For example, eggs were harvested for a time period of 12 hours. Ovisites were positioned in cages with gravid BSF female flies for 12 hours, before harvesting and collecting the ovisites filled with eggs. Alternatively, during a time frame of 12 hours, each hour an ovisite can be placed inside the cage and the ovisite which is placed an hour earlier is removed from the cage, such that twelve ovisites are obtained each comprising eggs with an age difference of 1 hour maximum, wherein the maximum age difference of the eggs is twelve hours for eggs in the first and twelfth ovisite. Directly after harvesting ovisites were stored in a first climate chamber at 30° C. and 80% RH for the first 42 hours post-harvest (youngest embryos: 42-43 hours of age; oldest embryos 54-55 hours of age), and subsequently, the eggs were transferred to a cold second climate chamber at 10° C. and 80% RH, for the next 24 hours. After this cooling step, eggs were returned to the first climate chamber and warmed to 30° C. and 80% RH, again, and kept at this conditions for the remainder of development (approximately three to six hours), until the eggs hatched at about 72 hours since eggs were laid. Influence of the cooling step on the number of hatching eggs was determined by comparing the number of hatched eggs after cooling and re-warming with the number of hatched eggs with a batch of eggs from ovisites that were harvested similarly after being positioned for 12 hours in the same cage with gravid BSF female flies, and were subsequently incubated uninterrupted at 30° C. and 80% RH for 58 hours, before counting of hatching eggs was started. The number of hatching eggs (emerging neonates from incubated control eggs and test eggs which were cooled) was counted during a period of 24 hours using a high-speed camera.

### Temperature Thresholds During the Cooling Step

For the cooling procedure, eggs were cooled at 1, 2, 3, 4, 5, 10, 12, 14 or 16° C., all for 24 hours. At all tested temperatures for the cooling step, eggs hatched to a certain extent once warmed to 30° C. again, if embryos were at least 36 hours of age at the start of the cooling and typically the eggs were about 40 hr of age, and/or if the eggs did no longer contain free yolk at the start of the cooling step and when the embryos reached a state of development before cooling started (as visualized by imaging using a photo camera as described), in which the transition from the rupture of the amnioserosa to completion of dorsal closure was established.

Cooling at 1° C. resulted in a lower extent of hatching of the eggs, compared to the tested higher temperatures for the cooling step. For pausing embryonic development, a suitable upper limit for the temperature during the cooling step is at lower than 14° C. such as 13° C. or 9° C.-12° C. At higher temperature for the cooling step, such as 14° C. or 16° C. tested, embryonic development is not halted (paused) during cooling, though still embryonic development is delayed. That is to say, in the experiment it was observed that embryos cooled to 14° C. or to 16° C. continue developing while at the cool temperature, whereas e.g. a cooling step at below 14° C. such as 12° C. or 10° C. stops embryonic development (as assessed from photos taken from the embryos at subsequent time points before, during and at the end of the cooling step of 24-120 hours), since warmed eggs hatched after a total time of about 58-60 hours at the warm temperature of 30° C., whereas cooling at 14° C. or 16° C. resulted in hatching of the eggs before the embryos were kept for in total 58 hours at 30° C. Indeed, continuing development of embryos while kept at 14° C. or 16° C. was also apparent from photos taken during the cooling step, compared to photos taken from embryos which were cooled for the same period at lower temperature, wherein embryos had the same age of about 40 hours at the start of the cooling period.

Cooling embryos at an age of 36 hours or older, such as about 40 hr and younger than 58 hr, at a cooling temperature of 7° C. - below 14° C., such as 10° C. is convenient for several reasons if delaying and timing the moment in time at which the eggs hatch is aimed for. The hatching success is the same or comparable when compared to control eggs which were not subjected to a cooling step of 24- 120 hours, such that the time between the depositing of the eggs (oviposite) and the hatching of the eggs is successfully expanded with 24-120 hours such as about 24 hours. The cooled embryos do not further develop during cooling when the cooling temperature is between 1° C. and a temperature below 14° C., such that a robust and constant process of timing of hatching of the eggs is established: here, after in total about 58 hours of incubation of the eggs at 30° C. and 80% RH, plus the time duration of the cooling step, if applied with eggs. Cooling the eggs from 30° C. to 10° C., and warming the eggs back to 30° C. after completion of the cooling step, demands relatively less energy, compared to cooling the eggs to a temperature below 10° C., while the hatching success with eggs cooled to 10° C. is the same as the hatching success obtained with eggs cooled to e.g. 4° C. - below 10° C. Furthermore, subjecting the embryos to a temperature during the cooling step which is closer to the warm temperature of 30° C., e.g. between 8° C. and below 14° C., may prevent delayed or damaged embryonic development when compared to eggs which are cooled from 30° C. down to a temperature below 8° C. or even 4° C. or below.

### Example 2

### Compression of Time Required for Hatching of Bsf Larvae - Controlling and Delaying the Time of Hatching

### Egg Collection - Embryo Incubation

BSF eggs were collected in ovisites, essentially according to the procedure outlined in Example 1. Ovisites comprised BSF eggs with a maximum age difference of 12 hours. A single ovisite was harvested from a cage with gravid female BSF flies after 12 hours, such that the age difference between the freshest egg in the ovisite and the first laid eggs in the ovisite is 12 hours at maximum.

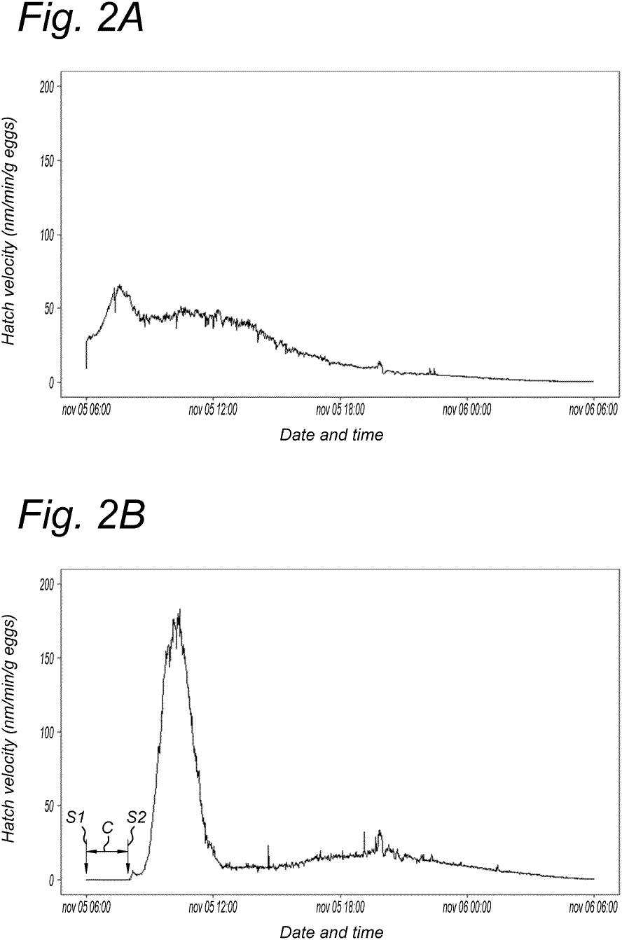

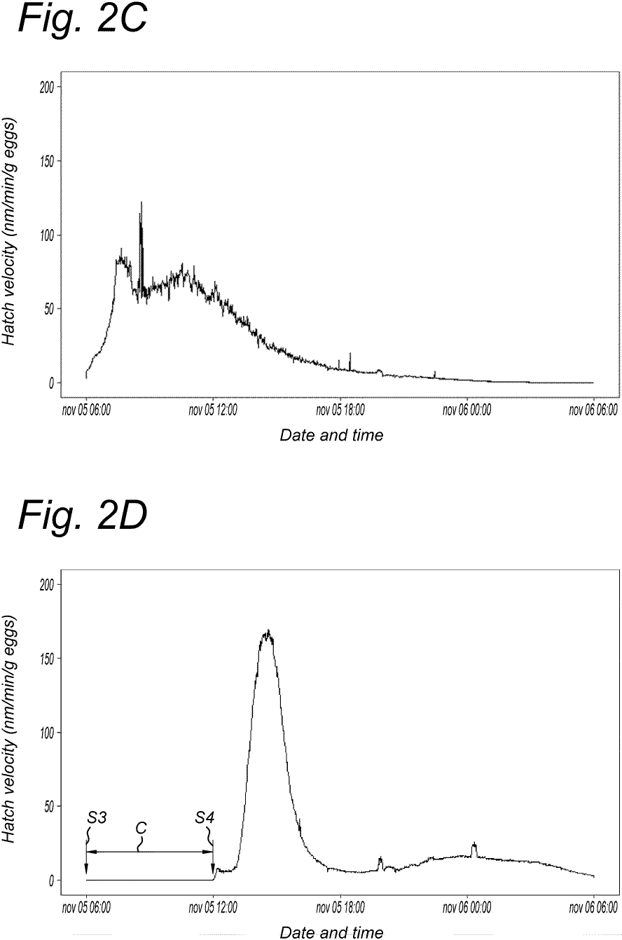

Embryos were incubated at 30° C. at 80% RH according to the method outlined in Example 1. All harvested eggs (in the ovisite or 12 ovisites) were kept in the climate room for 60 hours (relative to the oldest eggs; youngest eggs were thus kept at 30° C. for 48 hours post oviposition and harvesting of the ovisite). For the control measurement, 1586 grams of eggs were applied (FIG. 2A) or 857 grams of eggs (FIG. 2C). The cooling cycle was tested with 1115 grams of eggs (FIG. 2B, see description here below), or with 1103 grams of eggs (FIG. 2D).

Hatching success and rate under influence of a cooling step shortly before hatching of the eggs, at time point 60 hr post oviposition (relative to the oldest eggs; time point is 48 hr post oviposition relative to the freshest eggs) was assessed.

A first batch (Control batch 1; FIG. 2A) was then continuously kept at the incubation conditions (30° C. at 80% RH), starting from t = 60 hr onwards (relative to the oldest eggs), for 24 hr, and the extent of hatching of the eggs was assessed during these 24 hours (FIG. 2A, 1586 grams of eggs). Hatching eggs are observed for about 20 hours, with a first peak of hatching eggs around about 1.5 hours duration of the prolonged subjection of the eggs to incubation conditions, followed by an extended period of hatching eggs, declining from about 7 hours onwards. A second batch (1115 grams of eggs)) was subjected to a cooling step C lasting for 2 hours at 10° C. and 80% RH, starting at t = 60 hr (relative to the oldest eggs), indicated with S1, S2 in FIG. 2B, followed by warming the eggs back to 30° C. at 80% RH after the 2-hour cool period. Once warmed back to 30° C. at 80% RH, the extent of hatching of the eggs was assessed during these 22 hours, starting from time point S2 (end of cooling cycle C; FIG. 2B). Hatching eggs for Test Batch 1 are observed essentially for about 4.5-5 hours, starting after about 1-1.5 hours post cooling step, with the majority of hatching eggs before about 5 hours duration of the prolonged subjection of the eggs to incubation conditions, followed by a relatively minor second broad peak of hatching eggs, around 12 hours from the start of the second warm period after the cooling step.

In a separate experiment, embryos were incubated at 30° C. at 80% RH according to the method outlined in Example 1. Eggs were kept in the climate room for 58 hours (relative to the oldest eggs; youngest eggs were thus kept at 30° C. for 46 hours post oviposition and harvesting of the ovisite).

A third batch of 857 grams of BSF eggs was then continuously kept at the incubation conditions (30° C. at 80% RH), starting from t = 58 hr onwards (relative to the oldest eggs), for 24 hr, and the extent of hatching of the eggs was assessed during these 24 hours (FIG. 2C). Hatching eggs are observed for up to about 20 hours, with a first peak of hatching eggs around about 1.5 hours duration of the prolonged subjection of the eggs to incubation conditions, followed by an extended period of hatching eggs, declining from about 7 hours onwards. A fourth batch of 1103 grams of BSF eggs was subjected to a cooling step C of 6 hours at 10° C. and 80% RH, starting at t = 58 hr, at S3 (relative to the oldest eggs), followed by warming the eggs back to 30° C. at 80% RH after 6 hours of cooling at time point S4. Once warmed back to 30° C. at 80% RH, the extent of hatching of the eggs was assessed during these 18 hours, starting at S4 (FIG. 2D). Hatching eggs are again, similar to the eggs subjected for 2 hours to cooling, observed essentially for about 4.5-5 hours, now starting after about 1 hours post cooling step, with the majority of hatching eggs before about 5 hours duration of the prolonged subjection of the eggs to incubation conditions, followed by a relatively minor second broad peak of hatching eggs, around 18 hours from the start of the second warm period after the cooling step.

Compared to the control first batch of eggs (FIG. 2A), the 2-hours cooling step resulted in a compressed time window in which the majority of the eggs hatched, from a broad peak of hatching eggs lasting for about 18 hours to a narrow peak of hatching eggs for about 4.5-5 hours, whereas the yield within those 4.5-5 hours of the mean peak of hatching eggs after the cooling step is only about 1.6% lower than the total yield obtained with the eggs that were not subjected to the cooling step for 2 hours at 10° C. at 80% RH. Thus, at the expense of minimal loss in total yield of larvae (about 1.6%), the time to collect and obtain the larvae emerging from the eggs is reduced with at least a factor 3.

During the measurement of hatching extent of the eggs, temperature was 30° C. at 80% relative humidity.

Similarly, compared to the control second batch of eggs (FIG. 2C), the 6-hours cooling step resulted in a compressed time window in which the majority of the eggs hatched, from a broad peak of hatching eggs lasting for about 20 hours to a narrow peak of hatching eggs for about 4.5-5 hours, whereas the yield within those 4.5-5 hours of the mean peak of hatching eggs after the cooling step is only about 15.5% lower than the total yield obtained with the eggs that were not subjected to the cooling step for 6 hours at 10° C. at 80% RH. Thus, at the expense of minimal loss in total yield of larvae (about 15.5%), the time to collect and obtain the larvae emerging from the eggs is reduced with at least about a factor 4.

Note that for the controls, the eggs for the 2-hour cooling step were originating from the same cage (breed; parent BSF) as for the first control (FIG. 2B vs. FIG. 2A). The eggs for the 6-hour cooling step were originating from the same cage (breed; parent BSF) as for the second control (FIG. 2D vs. FIG. 2C).

### Example 3

Similar to Example 1 and 2, ovisites were positioned in cages comprising gravid BSF female flies. After 12 hours, ovisites were collected and either incubated for 58-60 hours at 30° C. at 80% RH in the dark (control; incubation time relative to the last deposited eggs, deposited shortly before collection of the ovisites), or incubated first for about 40 hours at 30° C. at 80% RH in the dark, followed by a 5-day incubation at 5° C. and 80% RH in the dark, subsequently followed by a further 18-20 hours incubation at 30° C. at 80% RH (test/5-days; incubation times are relative to the last deposited eggs, deposited shortly before collection of the ovisites). At the end of the incubation period (test/5-days) or three subsequent warm/cool/warm incubation periods (test/ 5-days), the extent of hatching eggs was assessed by counting living larvae emerging from the eggs comprised by the incubated ovisites. For both the control and for the test/5-days, eggs hatched and living larvae were obtained. These results show that a cooling period of at least five days is suitable for obtaining hatched BSF eggs thereafter.

### Example 4

### The Phases of Hermetia Illucens Embryo Development and Temperature’s Influence

The inventors apply newly established phases of embryo development for eggs of the Black Soldier Fly (Hermetia illucens; BSF), with a focus on BSF larvae development in the method of the invention. The egg stage of BSF was relatively unstudied until now. The inventors determined the phases of embryo development for BSF eggs and recognized the potential of the gained insights for e.g. delaying eggs by using a chilling protocol of the invention. The inventors found that when chilling eggs under specific conditions of temperature and relative humidity, such as preferably for 24 hours at 10° C. and 80% relative humidity, the development is paused for a full day. The inventors established that the gained insights present a window of opportunity for embryo ages, in particular ages of approximately 38 - 58 hours post oviposition, where the hatch success is the least affected (>80% hatching). The assessment, analysis and description of embryo development can be used as a reference tool for quality control purposes, according to the invention. The effect of treatments on eggs can be determined by identifying key processes in development. Furthermore, the chilling of embryos and thus pausing of development can be applied in a production setting.

A working knowledge of a species’ development is essential for its efficient cultivation. Characterized phases of developmental stages provide benchmarks for analysis of individuals in production and serve as starting points for deeper research into nuanced manipulation of development. Hermetia illucens (Diptera: Stratiomyidae), the black soldier fly (BSF), is an increasingly researched and cultivated insect species (Abd El-Hack et al., 2020; Mouithys-Mickalad et al., 2020; van Huis, 2013). Like all Diptera species, H. illucens undergoes complete metamorphosis through four life cycle stages as an egg, a larva, a pupa and an adult fly (Courtney et al., 2017).

Most life cycle characterization of H. illucens has been carried out on the larval stage. This bias is primarily because the larvae are processed into sellable product. Many studies assess the growth performance of H. illucens on novel feeds (Chia et al., 2018; Meneguz et al., 2018; Schreven et al., 2020). These studies provide life history detail that explains the rough development trajectory of larvae. More nuanced studies that provide clear information on physiological development are rarer (Holmes et al., 2012; Tomberlin et al., 2009). Pupal development is not as extensively studied, but (Barros-Cordeiro et al., 2014) provide a clear overview of the physical phases of development of the pupae. Physical developments of the adult fly have been examined in (Malawey et al., 2019). Most papers treating the development of adults provide some information about lifespan and fecundity (Booth and Sheppard, 1984; Hoc et al., 2019; Malawey et al., 2020; Sheppard et al., 2002), which are both connected to physiological developments in the fly. However, the underlying physiology of aging and fecundity is well understood. The embryological development of H. illucens is not well studied but of high interest. Production of eggs is a costly and complex activity, and once acquired, the eggs are vulnerable to different challenges from their environment, such as climate deficiencies. A detailed characterization of the development of the embryo of H. illucens can be useful in, for example, manipulation of embryo development and controlling of the life cycle of H. illucens.

Additionally, being able to control development of eggs is beneficial in the mass-rearing of H. illucens. Short-term storage of eggs will allow the biology to adapt to the production demands by synchronizing their development with the production process and working hours.

The inventors now assessed and established H. illucens (BSF) egg development through different phases, to infer the mechanisms and processes that are relevant. Particular attention is given to the time of each development and the role of temperature in development. Multiple experiments are performed to provide an overview of the variability of development that can be mapped to a production environment.

### Materials and Method

### Egg Collection

Eggs analysed by the inventors were collected in oviposition sites that have been made available to gravid black soldier flies for one hour in order to minimize the deviation in laying times between eggs. Each of the oviposition sites was presented to individual breeding cages of which the BSFs have a shared origin. This collection process resulted in eggs with a maximum age difference of one hour. Directly after collection, the eggs in their oviposition sites were stored in darkness in climate cabinets (Memmert ICH110) at 30° C. and 80% relative humidity (RH). The oviposition sites contained between 2 and 10 grams of eggs. One gram of eggs contains approximately 35.500 eggs (Booth and Sheppard, 1984).

### Imaging Embryo Development

Eggs were imaged to evaluate the embryo development. Eggs were continuously stored at 30° C. and 80% RH until imaging. Samples were taken from the oviposition sites and suspended in a droplet of water on microscope slides with cover glass for imaging and were discarded afterwards. Three clusters of eggs were taken from storage every hour, individual eggs were extracted from the cluster using a thin metal rod, and the embryo in the eggs were photographed. Every sample taken was from a unique cluster, to prevent artefacts due to mechanical damage. In combination, these images provide a full overview of the embryo development in steps of one hour resulting in 210 samples of eggs at 70 time points. Imaging was performed using a Euromex BioBlue.Lab trinocular microscope with Hayear (HY-3609B) camera mounted on it.

### Pausing Development

To pause embryo development within H. illucens eggs, the eggs are chilled. Chilled eggs, used to study the impact of pausing embryo development on hatch rate, were stored in a climate cabinet at 10° C. and 80% RH for 24 hours. The eggs were subjected to this chilling step at different egg ages (number of hours from egg deposition) for every three hours of embryo development (e.g. 0-1 hours old, 3-4 hours old, ..., 57-58 hours old), resulting in 120 samples of chilled eggs at 20 time intervals. The treated eggs have always been subjected to the control climate (30° C. and 80% RH) for a total of 60 hours, with the chilling step of 24 hours for every embryo age interval on a different moment between egg deposition and egg hatching. The control eggs were never subjected to the chilling step. This means that the control eggs have been in a climate cabinet for 60 hours and the treated eggs for a total of 84 hours. The eggs that were chilled have been imaged before and after the chilling period to establish that embryo development had been halted.

### Estimating Hatching Success

To estimate hatching success, the oviposition sites were attached to load cells and suspended above containers of water to capture larvae as they hatch and prevent fouling of the experimental setup. The load cells report the weight of the oviposition site every second and thus the weight over time was recorded. These load cells were straight bar load cells with a capacity of 100 grams. Electronics and software for logging was internally developed for this experiment. The difference between the starting weight and the weight recorded at the end of the experiment is primarily explained by the larvae hatching and falling off of the oviposition site, into the container of water. A part of the weight loss was also due to desiccation of eggs that did not hatch. The setup allowed for a maximum of 36 oviposition sites to be measured at a time, so each experiment consisted of a control of 12 oviposition sites and four times 6 oviposition sites for eggs that have been chilled at different egg ages. The success of hatching was estimated by taking the percent weight difference between start and finish of the experiment, subtracting the weight of the oviposition site beforehand, see equation 1 below.

$$\left(\frac{\left(W e i g h {t}_{b e f o r e} - W e i g h {t}_{o v i s i t e}\right) - \left(W e i g h {t}_{a f t e r} - W e i g h {t}_{o v i s i t e}\right)}{W e i g h {t}_{b e f o r e} - W e i g h {t}_{o v i s i t e}}\right) ∗ 100 \% \qquad \text{Equation (1):}$$

Percentage weight loss was determined as the decrease of the weight of the oviposition site containing the eggs, compared to the starting weight of said oviposition site. The mean of percent weight loss of the controls is set to 100%, assuming it is the maximum achievable weight loss with the current circumstances. Therefore, a weight loss of 100% for an experimental oviposition site means that as many eggs have hatched into viable larvae as within the controls. This weight loss percentage is referred to as relative hatch success.

### Data Handling

The hatch success data was analysed using the R statistical software (R Core Team, 2019). With the earth R package (Milborrow, 2019), we have used multivariate adaptive regression splines (MARS) to find the optimum egg age window of opportunity to pause egg development. MARS is a method of model fitting that is ideal for non-linear data. It automatically fits a model to the data. The model is a combination of linear models, where each linear model covers a section of the entire design space. MARS analysis aims to use the fewest number of local linear fits, while explaining the data well. The fitted MARS model has the relative hatch success as the dependent variable and the embryo age in hours as the independent variable.

### Results

### Embryo Development

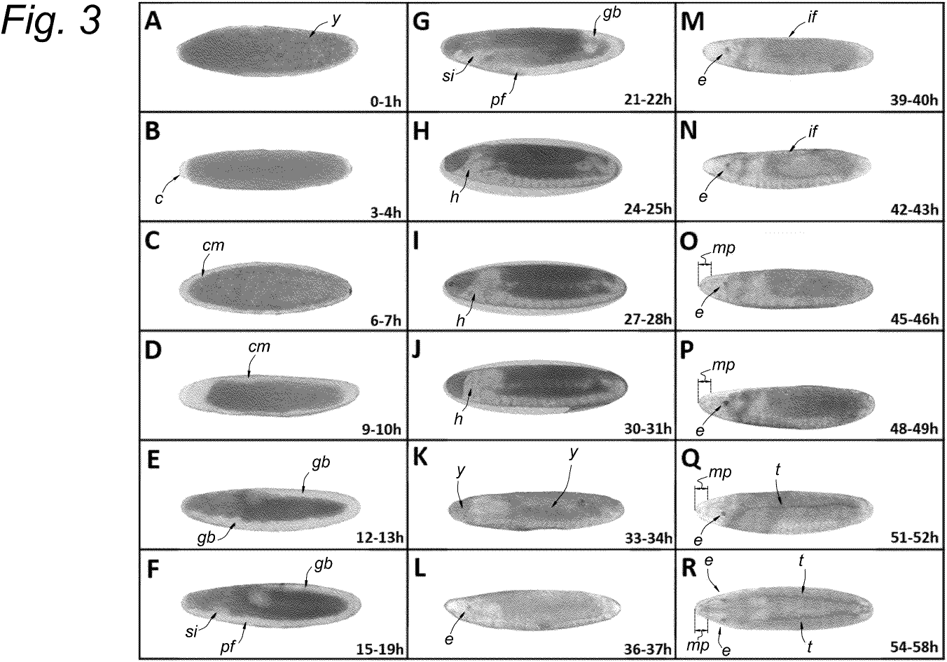

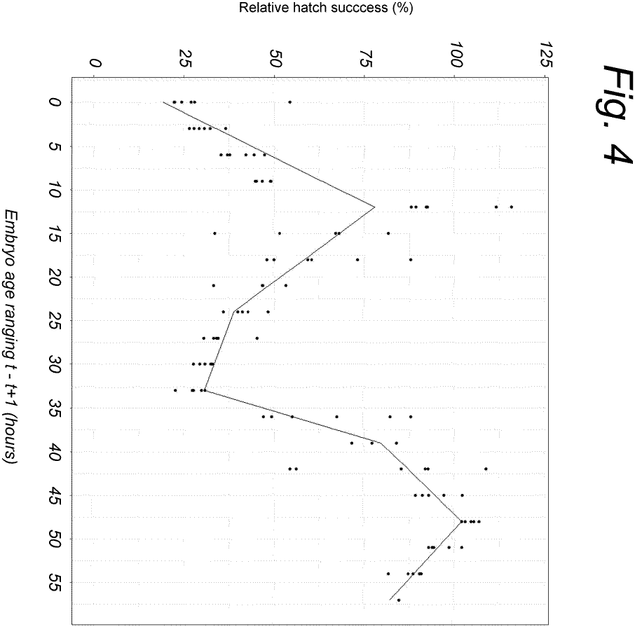

FIGS. 3A-R provides an overview of the phases of embryological development. While the sequence is fixed, the time that each stage takes to occur is highly dependent on temperature, and therefore single reference time in hours after laying can be given for any stage. It is noted that the first 0-1 hours of development occurred under breed climate settings due to the oviposition period of an hour. A freshly laid egg is an ellipse filled with yolk granules (FIG. 3A). The first visible changes in the BSF egg are the retraction of the anterior and posterior poles of the embryo from the chorion, which is the outer membrane of the egg shell (FIG. 3B). This first change enables a perceptive entomologist to distinguish fertilized eggs from unfertilized eggs. The retraction of the cytoplasm then leaves an empty space between the vitelline membrane, which is on the inside of the chorion, and the egg cytoplasm that contains the yolk. Next, cellular membranes are forming on the outside of the embryo resulting in a visible outer layer in a process called cellularization (FIG. 3C). Next, separation of the anterior blastoderm cells from the chorion and flattening occurs (FIG. 3D). Then, gastrulation takes place, where on the ventral side of the embryo the ectoderm, the outer germinal layer of the embryo invaginates. This invagination forms a mesodermal structure, which are germinal layers that are completely encapsulated within the embryo, called the germ band, and the phase of rapid germ band elongation starts (FIG. 3E). This is an extreme change in the morphology of the embryo. The next stage is the slow phase of germ band elongation (FIG. 3F), where the first parasegmental furrows in the germ band can also be observed. The stomodeal invagination is developing at this stage as well, which is part of the formation of the gut. After this stage, the germ band stays at its maximum length for approximately 6-7 hours and then starts to slowly retract (FIGS. 3G-3J), the head starts to form as well. In the next stage, the amnioserosa, which is an extraembryonic tissue that facilitates germ band extension, ruptures on the ventral side of the embryo and retracts to the dorsal side, which results in the migration of yolk from the cranial side of the embryo to the dorsal side (FIGS. 3K-3L), at this point, the eyes are clearly visible in the embryo. At the end of the amnioserosal retraction, dorsal closure starts, where the germ band fuses dorsally, resulting in visible segmentation (FIG. 3M). Segmentation becomes clearer when the intersegmental furrows appear (FIG. 3N) and the mouth parts start to form (FIGS. 3O-3P). Finally, we can see the formation of tracheae and when this has completed the egg is ready to hatch (FIGS. 3Q-3R).

Hatch success The results of the hatching success experiments for controls and treated eggs are summarised in the graph depicted in FIG. 4 (FIG. 4). The eggs have been subject to the chilling step for 24 hours, where chilling started at varying time points since oviposition with a 3 hour interval. It is assumed that the control eggs reflect the maximum achievable hatch success under the selected circumstances. Therefore, the mean hatching success of control eggs, which were not subjected to the chilling step, is set to 100%. The relative hatch success of the eggs that were chilled are corrected to percentage of the mean of the controls (equation 1.). The fitted MARS model had a high goodness of fit (R-squared = 0.81). The eggs that have been subjected to the chilling step at 0-1 hours or 33-34 hours old showed no signs of hatching upon visual inspection. The model also shows that these times have the lowest relative hatch success. It is concluded that chilling eggs at these developmental ages results in 100% loss of embryos. The measured weight loss of 20-30% by eggs at these times is most likely due to dehydration of the unviable eggs, from which no larvae emerged. Eggs that were chilled at ages between 3-4 and 30-31 hours old exhibited hatching, up to 25-50% of the maximum potential hatching when compared to the controls. Since dehydration of eggs in which embryos do not develop accounts for about 25% weight loss, it is estimated that when eggs are chilled starting at 3-31 hours after oviposition, at most 25% of recorded weight loss corresponds to viable embryos. This effect of dehydration is reduced as more eggs hatch, since hatched larvae are no longer contributing to the weight loss due to dehydration. Before hatching, these eggs also contribute to weight loss through metabolic activity and desiccation, however this is also the case for the controls.

Eggs subjected to chilling steps at 36-58 hours since oviposition yielded more efficient hatching. Median hatching success varied between 60% and 100% when compared to the controls. Hatching success was essentially the same as the control, for eggs that were subjected to the chilling step at 45, 48 or 51 hours since egg collection.

Knowledge on the embryo development of a species is used to monitor performance. In an experimental setting, the timing and phases of development is compared to the control.

A beneficial setting is covered by the method of the invention. With the method of the invention, the effect of chilling to pause embryo development across the embryo phases was tested, to find the most effective time window. The results of applying the method of the invention suggest that chilling of eggs is most viable after dorsal closure has occurred (approximately 36 hours of development at 30° C.). However, dorsal closure and less yolk does not explain why the embryo has such a high hatching success after being chilled for 24 hours at embryo age of 12-13 hours. This is just after the rapid phase of germ band extension, which is also a critical phase in the embryo development of insects.

The inventors established that there is a specific window of opportunity in which cooling is very effective. When the embryo age is not taken into account, the losses can be very high (Villazana and Alyokhin, 2019). Cryopreservation protocols that work for one Diptera species are not directly applicable to most other dipterans (Rajamohan et al., 2014; Wang et al., 2000).

An established window of opportunity for chilling of H. illucens embryos is between 38 and 58 hours after oviposition. The ability to pause the egg development of H. illucens provides more flexibility in a production environment. If a producer has excess eggs on one day, they can delay the excess for the day after. This grants the ability to cover for days with an egg yield which is below average. Overall, the stability of production can be assured when you are in control of egg hatching (Leopold, 1998; Rajamohan and Leopold, 2007).

### References

Abd El-Hack, M.E., Shafi, M.E., Alghamdi, W.Y., Abdelnour, S.A., Shehata, A.M., Noreldin, A.E., Ashour, E.A., Swelum, A.A., Al-Sagan, A.A., Alkhateeb, M., Taha, A.E., Abdel-Moneim, A.-M. E., Tufarelli, V., and Ragni, M., 2020. Black Soldier Fly ( Hermetia illucens) meal as a promising feed ingredient for poultry: A comprehensive review. Agriculture 10(8): 339. https://doi.org/10.3390/agriculture10080339

Barros-Cordeiro, K.B., Bao, S.N., and Pujol-Luz, J.R., 2014. Intra-puparial development of the black soldier-fly, Hermetia illucens. Journal of insect science 14(1). https://doi.org/10.1093/jis/14.1.83

Booth, D.C., and Sheppard, C., 1984. Oviposition of the Black Soldier Fly, Hermetia illucens (Diptera: Stratiomyidae): Eggs, masses, timing, and site characteristics. Environmental entomology 13(2): 421-423. https://doi.org/10.1093/ee/13.2.421

Chia, S.Y., Tanga, C.M., Osuga, I.M., Mohamed, S.A., Khamis, F.M., Salifu, D., Sevgan, S., Fiaboe, K.K.M., Niassy, S., Loon, J.J.A. van, Dicke, M., and Ekesi, S., 2018. Effects of waste stream combinations from brewing industry on performance of Black Soldier Fly, Hermetia illucens (Diptera: Stratiomyidae). PeerJ 6: e5885. https://doi.org/10.7717/peerj.5885

Courtney, G.W., Pape, T., Skevington, J.H., and Sinclair, B.J., 2017. Biodiversity of Diptera. Insect biodiversity: science and society 1: 229-278. https://doi.org/10.1002/9781118945568.ch9

Hoc, B., Noël, G., Carpentier, J., Francis, F., and Caparros Megido, R., 2019. Optimization of black soldier fly ( Hermetia illucens) artificial reproduction. PLoS One 14(4): e0216160. https://doi.org/10.1371/journal.pone.0216160

Holmes, L.A., Vanlaerhoven, S.L., and Tomberlin, J.K., 2012. Relative humidity effects on the life history of Hermetia illucens (Diptera: Stratiomyidae). Environmental entomology 41(4): 971-978. https://doi.org/10.1603/EN12054

Leopold, R.A., 1998. Cold storage of insects: using cryopreservation and dormancy as an aid to mass rearing. In Area-wide control of insect pests integrating the sterile insect and related nuclear and other techniques. FAO/IAEA International Conference, Penang, Malaysia (pp. 235-267). Available at: www.osti.gov/etdeweb/servlets/purl/683018

Malawey, A.S., Mercati, D., Love, C.C., and Tomberlin, J.K., 2019. Adult reproductive tract morphology and spermatogenesis in the Black Soldier Fly (Diptera: Stratiomyidae). Annals of the entomological society of America 112(6): 576-586. https://doi.org/10.1093/aesa/saz045

Malawey, A.S., Zhang, H., McGuane, A.S., Walsch, E.M., Rusch, T.W., Hjelmen, C.E., Delclos, P.J., Rangel, J., Zheng, L., Cai, M., Yu, Z., Tarone, A.M., Zhang, J., Tomberlin, J.K., 2020. Interaction of age and temperature on heat shock protein expression, sperm count, and sperm viability of the adult black soldier fly (Diptera: Stratiomyidae). Journal of insects as food and feed 1: 13. https://doi.org/10.3920/JIFF2020.0017

Meneguz, M., Schiavone, A., Gai, F., Dama, A., Lussiana, C., Renna, M., and Gasco, L., 2018. Effect of rearing substrate on growth performance, waste reduction efficiency and chemical composition of black soldier fly ( Hermetia illucens) larvae: Rearing substrate effects on performance and nutritional composition of black soldier fly. Journal of the science of food and agriculture 98(15): 5776-5784. https://doi.org/10.1002/jsfa.9127

Milborrow, S., 2019 Derived from mda:mars by Trevor Hastie and Rob Tibshirani. Uses Alan Miller’s Fortran utilities with Thomas Lumley’s leaps wrapper. earth: Multivariate Adaptive Regression Splines. R package version 5.1.1. https://CRAN.R-project.org/package=earth

Mouithys-Mickalad, A., Schmitt, E., Dalim, M., Franck, T., Tome, N.M., van Spankeren, M., Serteyn, D., and Paul, A., 2020. Black Soldier Fly ( Hermetia illucens) larvae protein derivatives: Potential to promote animal health. Animals 10(6): 941. https://doi.org/10.3390/ani10060941

Rajamohan, A., and Leopold, R.A., 2007. Cryopreservation of Mexican fruit flies by vitrification: Stage selection and avoidance of thermal stress. Cryobiology 54(1): 44-54. https://doi.org/10.1016/j.cryobiol.2006.10.192

Rajamohan, A., Rinehart, J.P., and Leopold, R.A., 2014. Cryopreservation of embryos of Lucilia sericata (Diptera: Calliphoridae). Journal of medical entomology 51(2): 360-367. https://doi.org/10.1603/ME13188

R Core Team (2019). R: A language and environment for statistical computing. R Foundation for Statistical Computing, Vienna, Austria. www.R-project.org.

RStudio Team (2020).RStudio: Integrated Development for R. RStudio, PBC, Boston, MA, www.rstudio.com.

Schreven, S.J.J., Yener, S., van Valenberg, H.J.F., Dicke, M., and van Loon, J.J.A., 2020. Life on a piece of cake: Performance and fatty acid profiles of black soldier fly larvae fed oilseed by-products. Journal of insects as food and feed 1-16. https://doi.org/10.3920/JIFF2020.0004

Sheppard, D.C., Tomberlin, J.K., Joyce, J.A., Kiser, B.C., and Sumner, S.M., 2002. Rearing methods for the Black Soldier Fly (Diptera: Stratiomyidae). Journal of medical entomology 39(4): 695-698. https://doi.org/10.1603/0022-2585-39.4.695

Tomberlin, J.K., Adler, P.H., and Myers, H.M., 2009. Development of the Black Soldier Fly (Diptera: Stratiomyidae) in relation to temperature. Environmental entomology 38(3): 5. https://doi.org/10.1603/022.038.0347

van Huis, A. 2013. Potential of insects as food and feed in assuring food security. Annual review of entomology 58: 563-583. https://doi.org/10.1146/annurev-ento-120811-153704

Villazana, J., and Alyokhin, A., 2019. Tolerance of immature Black Soldier Flies (Diptera: Stratiomyidae) to cold temperatures above and below freezing point. Journal of economic entomology 112(6): 2632-2637. https://doi.org/10.1093/jee/toz186

Wang, W.B., Leopold, R.A., Nelson, D.R., and Freeman, T.P., 2000. Cryopreservation of Musca domestica (Diptera: Muscidae) embryos. Cryobiology 41(2): 153-166. https://doi.org/10.1006/cryo.2000.2278

1. Method for controlling hatching of Black soldier fly eggs, comprising the steps of:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
bb) wherein incubation of Black soldier fly eggs at the first temperature allows Black soldier fly eggs to hatch from a time-point H1 onwards relative to the time point T0, during a time frame P2, when Black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermined second incubation period P4 ending at time point T3,
cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third incubation period P5 starting at T3 and ending at time point H2;
d) starting at T3 of step c), warming the cooled batch of eggs of step c) at the selected controlled first temperature of step b) for a third incubation period P5 ending at time point H2 at which H2 hatching of the Black soldier fly eggs in the provided batch of eggs starts; and
e) starting at H2, obtaining hatched batch of eggs during a time frame P6.

2-3. (canceled)

4. The method of claim 1 wherein time period P0 which lapsed at time point T1 relative to the time point T0 at which the first eggs of the batch of eggs are deposited, is at least 90 minutes and at longest the duration of time period P3, wherein P3 has the length of the time period starting at time point T0 and ending at time point H1 at which Black soldier fly eggs start hatching after incubation at the first temperature, according to step bb).

5. The method of claim 1, wherein time period P0 which lapsed at time point T1 relative to the time point T0 at which the first eggs of the batch of eggs are deposited, is at least 90 minutes and at longest 33.5 hours.

6. The method according to claim 1, wherein time period P0 which lapsed at time point T1 relative to the time point T0 at which the first eggs of the batch of eggs are deposited, is at least 34.5 hours and at longest 60 hours.

7. The method of claim 1 wherein the time period P3 of step b) is between 58 hours and 62 hours.

8. The method of claim 1 wherein the time frame P2 of step b) is between 12 hours and 20 hours starting from H1.

9. The method of claim 1, wherein the time frame P6 is shorter than the time frame P2, preferably P6 is shorter than 14 hours, more preferably between 3 hours and 11 hours, most preferably between 4 hours and 10 hours such as 4-6 hours.

10-13. (canceled)

14. The method according to claim 1, wherein the selected controlled second temperature is between 4° C. and below 14° C.

15. The method according to claim 1, wherein the selected first incubation period P1 is starting at time point T1, wherein T1 is selected from a time point between 1 hour relative to time point T0 at which the first eggs of the batch of eggs are deposited and time-point H1 at which Black soldier fly eggs start to hatch, according to step bb).

16. The method according to claim 1, wherein the selected first incubation period P1 is starting at time point T1, wherein T1 is selected from a time point between 3 hours relative to time point T0 at which the first eggs of the batch of eggs are deposited and 32 hours.

17. The method according to claim 1, wherein the selected controlled first temperature is between 25° C. and 35° C.

18. The method according to claim 1, wherein the selected first incubation period P1 and the third incubation period P5 together are the same or longer than time period P3.

19. The method according to claim 1, wherein during time frame P2 and/or during time frame P6, the temperature during hatching of the eggs is between 25° C. and 35° C.

20. (canceled)

21. The method according to claim 1, wherein the Black soldier fly eggs of the batch of eggs provided in step a) have a maximum age difference of between 30 minutes and 18 hours, wherein T1, T2, T3, H1 and H2 are relative to the time point T0 at which the first eggs of the batch of eggs are deposited.

22. (canceled)

23. The method according to claim 1, wherein time point T2 for starting cooling of the batch of eggs is at least 2 hours after time point T0 at which the first eggs of the batch of eggs are deposited, and at least 3 hours before yolk in the eggs is absent in the cranial side of embryos in the eggs or at least 3 hours after yolk in the eggs is absent in the cranial side of the embryos in the eggs.

24. The method according to claim 1, wherein time point T2 for starting cooling of the batch of eggs is at least 2 hours after time point T0 at which the batch of eggs is deposited, and at least 2 hours before dorsal closure in embryos in the eggs or at least 1 hour after dorsal closure in the embryos in the eggs.

25. (canceled)

26. The method according to claim 1, wherein in step c) T2 is selected from a time point between 25 hours and 0 hour before time point H1 of step bb).

27. Batch of Black soldier fly eggs comprising embryos and kept at a temperature of between 0° C. and below 14° C. with a relative humidity of 65% or higher, wherein the embryos are capable of developing into Black soldier fly neonates if the temperature is risen to 14° C. - 40° C. while the relative humidity remains 65% or higher.

28. (canceled)

29. The batch of Black soldier fly eggs comprising embryos according to claim 27, wherein the batch of eggs is provided with subsequently steps:
a) at a time point T1 providing a batch of Black soldier fly eggs for which selected lapsed time period P0 at time point T1 relative to a time point T0 at which first eggs of the batch of eggs are deposited, is known;
b) starting at T1, incubating the batch of eggs of step a) for a selected first incubation period P1 ending at a time point T2 relative to T1, at a selected controlled first temperature,
bb) wherein incubation of Black soldier fly eggs at the first temperature allows Black soldier fly eggs to hatch from a time-point H1 onwards relative to the time point T0, during a time frame P2, when black soldier fly eggs are incubated at the first temperature for a time period P3 starting at T0 and ending at H1;
c) starting at T2 of step b), cooling of the batch of eggs of step b) at a selected controlled second temperature lower than the first temperature, for a predetermind second incubation period P4 ending at time point T3,
cc) wherein starting at T2 cooling of Black soldier fly eggs at the second temperature allows Black soldier fly eggs to hatch from a time-point H2 onwards relative to the time point T0, when Black soldier fly eggs are first incubated at the first temperature for the time period P1 starting at T1 and ending at T2 according to step b) and subsequently incubated at the second temperature for the second incubation period P4 starting at T2 and ending at T3, followed by immediate warming the cooled Black soldier fly eggs at the selected controlled first temperature of step b) for a third invubation period P5 starting at T3 and ending at time point H2.

30. Batch of Black soldier fly eggs comprising embryos according to claim 27, wherein the embryos are capable of developing into Black soldier fly neonates if subjected to the following steps:
d) starting at T3, warming a cooled batch of eggs of step at a selected controlled first temperature of step for a third incubation period P5 ending at time point H2 at which H2 hatching of the Black soldier fly eggs in the provided batch of eggs starts: and
e) starting at H2, obtraining hatched batch of eggs during a time frame P6.
